SEQUENCE LISTING (55 sequences)

SEQ ID NO: 1 - protein, 36 aa, synthetic construct
NTGVSFANYTAHGSETAWADPLLTTSQLKALTNKDK

SEQ ID NO: 2 - protein, 37 aa, synthetic construct
  VARIANT 6: note = G or S
  VARIANT 18: note = S or A
  VARIANT 23: note = S or L
  VARIANT 24: note = L or V
  VARIANT 26: note = A or T
  VARIANT 27: note = T or S
  VARIANT 29: note = V or L
  VARIANT 36: note = D or N
LNTGVXFANYTAHGSETXWADPXXTXXQXKALTNKXK

SEQ ID NO: 3 - protein, 37 aa, synthetic construct
  VARIANT 8: note = V or A
FNGGISLXNYTGHGSETAWGTSHFGTTHVKQLTNSNQ

SEQ ID NO: 4 - protein, 23 aa, synthetic construct
  VARIANT 6: note = G or S
  VARIANT 18: note = S or A
  VARIANT 23: note = S or L
LNTGVXFANYTAHGSETXWADPX

SEQ ID NO: 5 - protein, 36 aa, synthetic construct
  VARIANT 5: note = G or S
  VARIANT 17: note = S or A
  VARIANT 22: note = S or L
  VARIANT 23: note = L or V
  VARIANT 25: note = A or T
  VARIANT 26: note = T or S
  VARIANT 28: note = V or L
  VARIANT 35: note = D or N
NTGVXFANYTAHGSETXWADPXXTXXQXKALTNKXK

SEQ ID NO: 6 - protein, 20 aa, synthetic construct
  VARIANT 2: note = G or S
  VARIANT 14: note = S or A
  VARIANT 19: note = S or L
  VARIANT 20: note = L or V
VXFANYTAHGSETXWADPXX

SEQ ID NO: 7 - protein, 22 aa, synthetic construct
LNTGVSFANYTAHGSETAWADP

SEQ ID NO: 8 - protein, 23 aa, synthetic construct
  VARIANT 8: note = V or A
FNGGISLXNYTGHGSETAWGTSH

SEQ ID NO: 9 - protein, 36 aa, synthetic construct
  VARIANT 7: note = V or A
NGGISLXNYTGHGSETAWGTSHFGTTHVKQLTNSNQ

SEQ ID NO: 10 - protein, 20 aa, synthetic construct
  VARIANT 4: note = V or A
ISLXNYTGHGSETAWGTSHF

SEQ ID NO: 11 - protein, 21 aa, synthetic construct
FNGGISLANYTGHGSETAWGT

SEQ ID NO: 12 - protein, 173 aa, synthetic construct
TLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFTNALLEETITAKGVRSPKAIRGRIQGTWRQKTVDLPAGTKYVAFRHFQSTDMFYIDLDEVEIKANGKR

SEQ ID NO: 13 - protein, 73 aa, synthetic construct
TLSESFENGIPASWKTIDADGDGHGWKPGNAPGIAGYNSNGCVYSESFGLGGIGVLTPDNYLITPALDLPNGG

SEQ ID NO: 14 - protein, 29 aa, synthetic construct
SNEFAPVQNLTGSSVGQKVTLKWDAPNGT

SEQ ID NO: 15 - protein, 57 aa, synthetic construct
ASYTYTVYRDGTKIKEGLTATTFEEDGVAAGNHEYCVEVKYTAGVSPKVCKDVTVEG

SEQ ID NO: 16 - protein, 86 aa, synthetic construct
ASYTYTVYRDGTKIKEGLTATTFEEDGVAAGNHEYCVEVKYTAGVSPKVCKDVTVEGSNEFAPVQNLTGSSVGQKVTLKWDAPNGT

SEQ ID NO: 17 - protein, 13 aa, synthetic construct
PNPNPNPNPNPGT

SEQ ID NO: 18 - protein, 99 aa, synthetic construct
ASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGT

SEQ ID NO: 19 - protein, 70 aa, synthetic construct
ASYTYTVYRDGTKIKEGLTATTFEEDGVAAGNHEYCVEVKYTAGVSPKVCKDVTVEGPNPNPNPNPNPGT

SEQ ID NO: 20 - protein, 29 aa, synthetic construct
SNEFAPVQNLTGSAVGQKVTLKWDAPNGT

SEQ ID NO: 21 - protein, 57 aa, synthetic construct
  VARIANT 30: note = A or T
SDYTYTVYRDGTKIKEGLTATTFEEDGVAXGNHEYCVEVKYTAGVSPKVCKDVTVEG

SEQ ID NO: 22 - protein, 15 aa, synthetic construct
PNPNPNPNPNPNPGT

SEQ ID NO: 23 - protein, 101 aa, synthetic construct
  VARIANT 30: note = A or T
SDYTYTVYRDGTKIKEGLTATTFEEDGVAXGNHEYCVEVKYTAGVSPKVCKDVTVEGSNEFAPVQNLTGSAVGQKVTLKWDAPNGTPNPNPNPNPNPNPGT

SEQ ID NO: 24 - protein, 165 aa, synthetic construct
AEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDM

SEQ ID NO: 25 - protein, 127 aa, synthetic construct
WGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDM

SEQ ID NO: 26 - protein, 271 aa, synthetic construct
AEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEVEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGT

SEQ ID NO: 27 - protein, 242 aa, synthetic construct
AEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEVEDDSPASYTYTVYRDGTKIKEGLTATTFEEDGVAAGNHEYCVEVKYTAGVSPKVCKDVTVEGPNPNPNPNPNPGT

SEQ ID NO: 28 - protein, 258 aa, synthetic construct
AEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEVEDDSPASYTYTVYRDGTKIKEGLTATTFEEDGVAAGNHEYCVEVKYTAGVSPKVCKDVTVEGSNEFAPVQNLTGSSVGQKVTLKWDAPNGT

SEQ ID NO: 29 - protein, 343 aa, synthetic construct
NTGVSFANYTAHGSETAWADPLLTTSQLKALTNKDKAEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEVEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTNTGVSFANYTAHGSETAWADPLLTTSQLKALTNKDK

SEQ ID NO: 30 - protein, 307 aa, synthetic construct
NTGVSFANYTAHGSETAWADPLLTTSQLKALTNKDKAEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEVEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGT

SEQ ID NO: 31 - protein, 379 aa, synthetic construct
NTGVSFANYTAHGSETAWADPLLTTSQLKALTNKDKAEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEVEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTNTGVSFANYTAHGSETAWADPLLTTSQLKALTNKDKNTGVSFANYTAHGSETAWADPLLTTSQLKALTNKDK

SEQ ID NO: 32 - protein, 343 aa, synthetic construct
  VARIANT 314: note = V or A
NTGVSFANYTAHGSETAWADPLLTTSQLKALTNKDKAEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEVEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTNGGISLXNYTGHGSETAWGTSHFGTTHVKQLTNSNQ

SEQ ID NO: 33 - protein, 345 aa, synthetic construct
NTGVSFANYTAHGSETAWADPLLTTSQLKALTNKDKKLNTGVSFANYTAHGSETAWADPLLTTSQLKALTNKDKAEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEVEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGT

SEQ ID NO: 34 - protein, 427 aa, synthetic construct
NTGVSFANYTAHGSETAWADPLLTTSQLKALTNKDKDSSGASNTGVSFANYTAHGSETAWADPLLTTSQLKALTNKDKAEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEVEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTNTGVSFANYTAHGSETAWADPLLTTSQLKALTNKDKKLDSSGNTGVSFANYTAHGSETAWADPLLTTSQLKALTNKDK

SEQ ID NO: 35 - protein, 342 aa, synthetic construct
NTGVSFANYTAHGSETAWADPLLTTSQLKALTNKDKWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEVEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFENGIPASWKTIDADGDGHGWKPGNAPGIAGYNSNGCVYSESFGLGGIGVLTPDNYLITPALDLPNGG

SEQ ID NO: 36 - protein, 380 aa, synthetic construct
NTGVSFANYTAHGSETAWADPLLTTSQLKALTNKDKAEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEVEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFENGIPASWKTIDADGDGHGWKPGNAPGIAGYNSNGCVYSESFGLGGIGVLTPDNYLITPALDLPNGG

SEQ ID NO: 37 - protein, 442 aa, synthetic construct
NTGVSFANYTAHGSETAWADPLLTTSQLKALTNKDKWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEVEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFENGIPASWKTIDADGDGHGWKPGNAPGIAGYNSNGCVYSESFGLGGIGVLTPDNYLITPALDLPNGGKLTFWVCAQDANYASEHYAVYASSTGNDASNFTNALLEETITAKGVRSPKAIRGRIQGTWRQKTVDLPAGTKYVAFRHFQSTDMFYIDLDEVEIKANGKR

SEQ ID NO: 38 - protein, 378 aa, synthetic construct
NTGVSFANYTAHGSETAWADPLLTTSQLKALTNKDKWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEVEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFENGIPASWKTIDADGDGHGWKPGNAPGIAGYNSNGCVYSESFGLGGIGVLTPDNYLITPALDLPNGGNTGVSFANYTAHGSETAWADPLLTTSQLKALTNKDK

SEQ ID NO: 39 - protein, 416 aa, synthetic construct
NTGVSFANYTAHGSETAWADPLLTTSQLKALTNKDKAEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEVEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFENGIPASWKTIDADGDGHGWKPGNAPGIAGYNSNGCVYSESFGLGGIGVLTPDNYLITPALDLPNGGNTGVSFANYTAHGSETAWADPLLTTSQLKALTNKDK

SEQ ID NO: 40 - protein, 500 aa, synthetic construct
NTGVSFANYTAHGSETAWADPLLTTSQLKALTNKDKDSSGASNTGVSFANYTAHGSETAWADPLLTTSQLKALTNKDKAEGSREVKRIGDGLFVTIEPANDVRANEAKVVLAADNVWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEVEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGTTLSESFENGIPASWKTIDADGDGHGWKPGNAPGIAGYNSNGCVYSESFGLGGIGVLTPDNYLITPALDLPNGGNTGVSFANYTAHGSETAWADPLLTTSQLKALTNKDKKLDSSGNTGVSFANYTAHGSETAWADPLLTTSQLKALTNKDK

SEQ ID NO: 41 - protein, 268 aa, synthetic construct
NTGVSFANYTAHGSETAWADPLLTTSQLKALTNKDKWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEVEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPG

SEQ ID NO: 42 - protein, 304 aa, synthetic construct
NTGVSFANYTAHGSETAWADPLLTTSQLKALTNKDKWGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEVEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGNTGVSFANYTAHGSETAWADPLLTTSQLKALTNKDK

SEQ ID NO: 43 - protein, 233 aa, synthetic construct
WGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEVEDDSPASYTYTVYRDGTKIKEGLTATTFEEDGVAAGNHEYCVEVKYTAGVSPKVCKDVTVEGSNEFAPVQNLTGSSVGQKVTLKWDAPNGTPNPNPNPNPNPGT

SEQ ID NO: 44 - protein, 204 aa, synthetic construct
WGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEVEDDSPASYTYTVYRDGTKIKEGLTATTFEEDGVAAGNHEYCVEVKYTAGVSPKVCKDVTVEGPNPNPNPNPNPGT

SEQ ID NO: 45 - protein, 220 aa, synthetic construct
WGDNTGYQFLLDADHNTFGSVIPATGPLFTGTASSNLYSANFEYLIPANADPVVTTQNIIVTGQGEVVIPGGVYDYCITNPEPASGKMWIAGDGGNQPARYDDFTFEAGKKYTFTMRRAGMGDGTDMEVEDDSPASYTYTVYRDGTKIKEGLTATTFEEDGVAAGNHEYCVEVKYTAGVSPKVCKDVTVEGSNEFAPVQNLTGSSVGQKVTLKWDAPNGT

SEQ ID NO: 46 - protein, 7 aa, synthetic construct
EVEDDSP

SEQ ID NO: 47 - protein, 5 aa, synthetic construct
GSGGS

SEQ ID NO: 48 - protein, 4 aa, synthetic construct
GGGS

SEQ ID NO: 49 - protein, 5 aa, synthetic construct
GGGGS

SEQ ID NO: 50 - protein, 4 aa, synthetic construct
DSSG

SEQ ID NO: 51 - protein, 6 aa, synthetic construct
DSSGAS

SEQ ID NO: 52 - protein, 6 aa, synthetic construct
KLDSSG

SEQ ID NO: 53 - protein, 6 aa, synthetic construct
GSGSGS

SEQ ID NO: 54 - protein, 22 aa, synthetic construct
GSGSGSGSGSGSGSGSGSGSGS

SEQ ID NO: 55 - protein, 15 aa, synthetic construct
GGGGSGGGGSGGGGS

(19) United States
(12) Patent Application Publication
(10) Pub. No.: US 2026/0317747 A1
(43) Pub. Date: Oct. 15, 2026

(54) CHIMERIC POLYPEPTIDES

(71) Applicant: DENTERIC PTY LTD, Melbourne, Victoria (AU)

(72) Inventors: Eric Charles Reynolds, Melbourne, Victoria (AU); Nada Slakeski, Melbourne, Victoria (AU); Neil O'Brien-Simpson, Melbourne, Victoria (AU)

(73) Assignee: DENTERIC PTY LTD, Melbourne, Victoria (AU)

(21) Appl. No.: 18/730,629

(22) PCT Filed: Jan. 20, 2023
(86) PCT No.: PCT/AU2023/050031

§ 371 Date: Jul. 19, 2024

(30) Foreign Application Priority Data
Jan. 20, 2022 (AU) 2022900102

(51) Int. Cl. C12N 9/52; A61K 39/02; A61P 31/04

(52) U.S. Cl. CPC C12N 9/52; A61K 39/0216; A61P 31/04; C12Y 304/22037; C12Y 304/22047

(57) ABSTRACT

A chimeric or fusion protein for inducing an immune response to P. gingivalis, the protein comprising a first polypeptide linked to a second polypeptide, wherein: A) the first polypeptide comprises or consists of an amino acid sequence of the active site of an Arg- or Lys-gingipain of P. gingivalis, or a sequence that is at least 80% identical thereto; and B) the second polypeptide comprises or consists of an amino acid sequence of an adhesin domain of an Arg- or Lys-gingipain of P. gingivalis; and wherein the second polypeptide: a) does not comprise part or all of the sequence of a cleaved adhesin domain (CAD), preferably; and/or b) comprises an amino acid sequence corresponding substantially to the full length of the DUF2436 domain of an Arg- or Lys-gingipain, or a sequence at least 80% identical thereto.

### FIELD OF THE INVENTION

The invention relates to chimeric polypeptides that are useful for inducing an immune response to P. gingivalis, compositions comprising same and uses thereof for the prevention and treatment of P. gingivalis-related conditions and diseases.

### RELATED APPLICATION

This application is the U.S. National Stage of International Application No. PCT/AU2023/050031 filed Jan. 20, 2023, and claims priority from Australian provisional application AU 2022900102 filed Jan. 20, 2022, the entire contents of which are hereby incorporated by reference.

### SEQUENCE LISTING

The instant application contains a Sequence Listing which has been submitted electronically in XML file format and is hereby incorporated by reference in its entirety. Said XML copy, created on Sep. 12, 2024, is named 53239434AZD—updated—US18_730629.xml and is 67,129 bytes in size.

### BACKGROUND OF THE INVENTION

If dental plaque is left to accumulate around the tooth at the gingival (gum) margin this causes gingival inflammation (gingivitis). Chronic gingivitis can allow the emergence of a periodontal pathogen Porphyromonas gingivalis (P. gingivalis) at the base of a periodontal pocket to result in a chronic infection and the development of severe disease. This severe form of periodontal disease is called periodontitis and can lead to tooth loss in an approach by the immune system to eliminate the infection.

Chronic periodontitis is an inflammatory disease of the supporting tissues of the teeth leading to resorption of alveolar bone and eventual tooth loss. The disease is a major public health problem in all societies and is estimated to affect up to 30% of the adult population with severe forms affecting 12-15% of the adult population.

One in three adults have moderate to severe periodontitis. From epidemiological surveys, periodontitis has been linked to an increased risk of inflammatory diseases including cardiovascular diseases, certain cancers, preterm birth, rheumatoid arthritis and dementia. More recent research has linked chronic infection by P. gingivalis with dementia and rheumatoid arthritis. For example, in one study, 96% of Alzheimer's disease (AD) brain samples showed the presence of P. gingivalis. Another study shows that chronic oral infection of mice with P. gingivalis resulted in the brain plaques associated with AD in humans and that the P. gingivalis proteases could cleave amyloid precursor and tau proteins to form the plaques and tangles associated with AD.

A number of virulence factors have been reported to contribute to the pathogenicity of P. gingivalis including; LPS, fimbriae, hemagglutinin, hemolysin and extracellular hydrolytic enzymes (especially the Arg-X and Lys-X specific proteinases), otherwise known as “P. gingivalis gingipains”.

The magnitude of the public health problem is such that there is a need for a vaccine that provides a strong protective response to P. gingivalis infection and means for providing same.

One problem has been that it is not clear how to obtain a strong protective response to P. gingivalis infection where there are a plethora of virulence factors to select from.

There is currently no commercially approved vaccine for use in preventing or reducing the incidence and/or severity of P. gingivalis infection or for treating P. gingivalis infection and disease in subjects.

There is therefore a need for alternative and/or improved approaches for the design and manufacture of P. gingivalis vaccines, and alternative and/or improved vaccines produced from P. gingivalis.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present invention provides a chimeric or fusion protein for inducing an immune response to P. gingivalis, the protein comprising a first polypeptide and a second polypeptide, wherein:
A) the first polypeptide comprises or consists of an amino acid sequence of the active site of an Arg- or Lys-gingipain of P. gingivalis, or a sequence that is at least 80% identical thereto; and
B) the second polypeptide comprises or consists of an amino acid sequence of an adhesin domain of an Arg- or Lys-gingipain of P. gingivalis;
wherein the second polypeptide comprises the sequence of one or more adhesin binding motifs (ABMs), preferably wherein the ABMs correspond to some or all of the ABMs between the DUF2436 domain and cleaved adhesin domain (CAD) of a P. gingivalis gingipain;
preferably wherein the one or more ABMs comprise the sequence as set forth in SEQ ID NO: 15 or 21 (ABM2), and/or SEQ ID NO: 17 or 22 (AMB3) or sequences at least 80% identical thereto,
more preferably wherein the one or more ABMs comprise the sequence as set forth in SEQ ID NO: 15 or 21 (ABM2), SEQ ID NO: 14 or 20 (ABM1) and/or SEQ ID NO: 17 or 22 (ABM3) or sequences at least 80% identical thereto,
most preferably wherein the one or more ABMs comprise the sequence set forth in any of SEQ ID NO: 16 or SEQ ID NO: 18 or SEQ ID NO: 19 or SEQ ID NOs: 23 or 26, 27, 28, 43, 44 or 45 or sequences at least 80% identical thereto;
and wherein the second polypeptide:
a) does not comprise: a sequence of a cleaved adhesin domain (CAD) or part thereof, preferably a sequence of a CAD having a sequence set forth in SEQ ID NOs: 12 or 13 or sequences at least 80% identical thereto; and/or
b) comprises an amino acid sequence corresponding substantially to the full length of the DUF2436 domain of an Arg- or Lys-gingipain, preferably as set forth in SEQ ID NO: 24, or a sequence at least 80% identical thereto.

Preferably, the chimeric or fusion protein comprises one or more further polypeptides that comprise or consist of an amino acid sequence of the active site of an Arg- or Lys-gingipain of P. gingivalis, or sequences that are at least 80% identical thereto. The one or more further polypeptides comprising or consisting of the active site of an Arg- or Lys-gingipain of P. gingivalis may be located N-terminally to the first polypeptide, C-terminally to the first polypeptide, N-terminally to the second polypeptide or C-terminally to the second polypeptide. In certain embodiments, at least two further polypeptides that comprise or consist of an amino acid sequence of the active site of an Arg- or Lys-gingipain of P. gingivalis, or sequences that are at least 80% identical thereto may be present. In such embodiments, the two further polypeptides may be located N-terminally to the second polypeptide, C-terminally to the second polypeptide, or N and C terminally to the second polypeptide.

The one or more further polypeptides may be linked to the first or second polypeptide of the chimeric or fusion protein, preferably via a linker of no more than 50 amino acids, or directly linked to the first or second polypeptide.

In any embodiment, the first polypeptide comprises or consists of an amino acid sequence selected from the group of: SEQ ID NOs: 1 to 11, or sequences at least 80%, 81%, 82%, 83%, 84%, 85%, 86%, 87%, 88%, 90%, 91%, 92%, 93%, 94%, 95%, 96%, 97%, 98%, or 99% identical thereto.

The one or more further polypeptides preferably comprise or consist of an amino acid sequence selected from the group of: SEQ ID NOs: 1 to 11, or sequences at least 80%, 81%, 82%, 83%, 84%, 85%, 86%, 87%, 88%, 90%, 91%, 92%, 93%, 94%, 95%, 96%, 97%, 98%, or 99% identical thereto.

In any embodiment, the first polypeptide and the further polypeptide that comprise or consist of an amino acid sequence of the active site of an Arg- or Lys-gingipain of P. gingivalis, comprise or consist of an amino acid sequence that is at least 80%, 81%, 82%, 83%, 84%, 85%, 86%, 87%, 88%, 90%, 91%, 92%, 93%, 94%, 95%, 96%, 97%, 98%, or 99% identical, or wherein the amino acid sequences are identical. The first polypeptide and the further polypeptide may be derived from the active site of heterologous gingipains (e.g., from gingipains of different strains of P. gingivalis). The first polypeptide and the further polypeptide may have an amino acid sequence that is derived from different gingipains (e.g., wherein one of the polypeptides has an amino acid sequence of an active site from a Kgp and the other polypeptide has an amino acid sequence of an active site from an Rgp; or alternatively wherein one of the polypeptides has an amino acid sequence of an active site from a RgpA and the other polypeptide has an amino acid sequence of an active site from an RgpB).

In a first aspect, the present invention provides a chimeric or fusion protein for inducing an immune response to P. gingivalis, the protein comprising a first polypeptide and a second polypeptide, wherein:
A) the first polypeptide comprises or consists of an amino acid sequence of the active site of an Arg- or Lys-gingipain of P. gingivalis, or a sequence that is at least 80%, 81%, 82%, 83%, 84%, 85%, 86%, 87%, 88%, 90%, 91%, 92%, 93%, 94%, 95%, 96%, 97%, 98%, or 99% identical thereto;
B) the second polypeptide comprises or consists of an amino acid sequence of an adhesin domain of an Arg- or Lys-gingipain of P. gingivalis;
wherein the second polypeptide comprises the sequence of one or more adhesin binding motifs (ABMs), preferably wherein the ABMs correspond to some or all of the ABMs between the DUF2436 domain and cleaved adhesin domain (CAD) of a P. gingivalis gingipain;
preferably wherein the one or more ABMs comprise the sequence as set forth in SEQ ID NO: 15 or 21 (ABM2), and/or SEQ ID NO: 17 or 22 (ABM3) or sequences at least 80%, 81%, 82%, 83%, 84%, 85%, 86%, 87%, 88%, 90%, 91%, 92%, 93%, 94%, 95%, 96%, 97%, 98%, or 99% identical thereto,
more preferably wherein the one or more ABMs comprise the sequence as set forth in SEQ ID NO: 15 or 21 (ABM2), SEQ ID NO: 14 or 20 (ABM1) and/or SEQ ID NO: 17 or 22 (ABM3) or sequences at least 80%, 81%, 82%, 83%, 84%, 85%, 86%, 87%, 88%, 90%, 91%, 92%, 93%, 94%, 95%, 96%, 97%, 98%, or 99% identical thereto,
most preferably wherein the one or more ABMs comprise the sequence set forth in any of SEQ ID NO: 16 or SEQ ID NO: 18 or SEQ ID NO: 19 or SEQ ID NOs: 23 or 26, 27, 28, 43, 44 or 45 or a sequence at least 80%, 81%, 82%, 83%, 84%, 85%, 86%, 87%, 88%, 90%, 91%, 92%, 93%, 94%, 95%, 96%, 97%, 98%, or 99% identical thereto;
and wherein the second polypeptide:
a) does not comprise a sequence of a cleaved adhesin domain (CAD), the CAD preferably having the sequence set forth in SEQ ID NO: 12 or 13 or sequences at least 80%, 81%, 82%, 83%, 84%, 85%, 86%, 87%, 88%, 90%, 91%, 92%, 93%, 94%, 95%, 96%, 97%, 98%, or 99% identical thereto;
optionally, wherein the chimeric or fusion protein comprises one or more further polypeptides that comprise or consist of an amino acid sequence of the active site of an Arg- or Lys-gingipain of P. gingivalis, or sequences that are at least 80% identical thereto.

The one or more further polypeptides comprising or consisting of the active site of an Arg- or Lys-gingipain of P. gingivalis may be located N-terminally to the first polypeptide, C-terminally to the first polypeptide, N-terminally to the second polypeptide of C-terminally to the second polypeptide. The one or more further polypeptides may be linked to the first or second polypeptide of the chimeric or fusion protein, preferably via a linker of no more than 50 amino acids, or directly linked to the first or second polypeptide.

In any embodiment, the first polypeptide comprises or consists of an amino acid sequence selected from the group of: SEQ ID NOs: 1 to 11, or sequences at least 80%, 81%, 82%, 83%, 84%, 85%, 86%, 87%, 88%, 90%, 91%, 92%, 93%, 94%, 95%, 96%, 97%, 98%, or 99% identical thereto.

The one or more further polypeptides preferably comprise or consist of an amino acid sequence selected from the group of: SEQ ID NOs: 1 to 11, or sequences at least 80%, 85%, 90%, 91%, 92%, 93%, 94%, 95%, 96%, 97%, 98%, or 99% identical thereto.

In any embodiment of the first aspect of the invention, the first polypeptide and the further polypeptide that comprise or consist of an amino acid sequence of the active site of an Arg- or Lys-gingipain of P. gingivalis, comprise or consist of an amino acid sequence that is at least 80%, 81%, 82%, 83%, 84%, 85%, 86%, 87%, 88%, 90%, 91%, 92%, 93%, 94%, 95%, 96%, 97%, 98%, or 99% identical, or wherein the amino acid sequences are identical. The first polypeptide and the further polypeptide may be derived from the active site of heterologous gingipains (e.g., from gingipains of different strains of P. gingivalis). The first polypeptide and the further polypeptide may have an amino acid sequence that is derived from different gingipains (e.g., wherein one of the polypeptides has an amino acid sequence of an active site from a Kgp and the other polypeptide has an amino acid sequence of an active site from an Rgp; or alternatively wherein one of the polypeptides has an amino acid sequence of an active site from a RgpA and the other polypeptide has an amino acid sequence of an active site from an RgpB).

In particularly preferred embodiments of the first aspect of the invention, the chimeric or fusion protein comprises or consists of an amino acid sequence as set forth in any one of 41, or 42 or sequences at least 80%, 81%, 82%, 83%, 84%, 85%, 86%, 87%, 88%, 90%, 91%, 92%, 93%, 94%, 95%, 96%, 97%, 98%, or 99% thereto.

In a second aspect, the present invention provides a chimeric or fusion protein for inducing an immune response to P. gingivalis, the protein comprising a first polypeptide linked to a second polypeptide, wherein:
A) the first polypeptide comprises or consists of an amino acid sequence of the active site of an Arg- or Lys-gingipain of P. gingivalis, or a sequence that is at least 80% identical thereto; and
B) the second polypeptide comprises or consists of an amino acid sequence of an adhesin domain of an Arg- or Lys-gingipain of P. gingivalis;
wherein the second polypeptide comprises the sequence of one or more adhesin binding motifs (ABMs), preferably wherein the ABMs correspond to some or all of the ABMs between the DUF2436 domain and cleaved adhesin domain (CAD) of a P. gingivalis gingipain;
preferably wherein the one or more ABMs comprise the sequence as set forth in SEQ ID NO: 15 or 21 (ABM2), and/or SEQ ID NO: 17 or 22 (ABM3) or sequences at least 80% identical thereto,
more preferably wherein the one or more ABMs comprise the sequence as set forth in SEQ ID NO: 15 or 21 (ABM2), SEQ ID NO: 14 or 20 (ABM1) and/or SEQ ID NO: 17 or 22 (ABM3) or sequences at least 80% identical thereto,
most preferably wherein the one or more ABMs comprise the sequence set forth in any of SEQ ID NO: 16 or SEQ ID NO: 18, 19 or 23, or SEQ ID NO: 26, 27, 28 or a sequence at least 80% identical thereto;
and wherein the second polypeptide:
a) does not comprise: a sequence of a cleaved adhesin domain (CAD) or part thereof, the CAD preferably having a sequence set forth in SEQ ID NOs: 12 or 13 or sequences at least 80% identical thereto; and
b) comprises an amino acid sequence corresponding substantially to the full length of the DUF2436 domain of an Arg- or Lys-gingipain, preferably as set forth in SEQ ID NO: 24, or a sequence at least 80% identical thereto.

As used, herein, an amino acid sequence corresponding substantially to the full length of the DUF2436 domain, or a sequence at least 80% identical thereto, refers to a sequence that is at least 80%, 81%, 82%, 83%, 84%, 85%, 86%, 87%, 88%, 90%, 91%, 92%, 93%, 94%, 95%, 96%, 97%, 98%, or 99% of the length of a DUF2436 domain of an Arg- or Lys-gingipain.

In a preferred embodiment, the amino acid sequence of the DUF2436 domain of an Arg- or Lys-gingipain is the sequence set forth in SEQ ID NO: 24 or is a sequence at least 80%, 81%, 82%, 83%, 84%, 85%, 86%, 87%, 88%, 90%, 91%, 92%, 93%, 94%, 95%, 96%, 97%, 98%, or 99% identical thereto.

Most preferably, the second polypeptide comprises or consists of the sequence set forth in SEQ ID NO: 26, 27, or 28 or a sequence at least 80%, 81%, 82%, 83%, 84%, 85%, 86%, 87%, 88%, 90%, 91%, 92%, 93%, 94%, 95%, 96%, 97%, 98%, or 99% identical thereto.

Preferably, the chimeric or fusion protein comprises one or more further polypeptides that comprise or consist of an amino acid sequence of the active site of an Arg- or Lys-gingipain of P. gingivalis, or sequences that are at least 80%, 81%, 82%, 83%, 84%, 85%, 86%, 87%, 88%, 90%, 91%, 92%, 93%, 94%, 95%, 96%, 97%, 98%, or 99% identical thereto. The one or more further polypeptides comprising or consisting of the active site of an Arg- or Lys-gingipain of P. gingivalis may be located N-terminally to the first polypeptide, C-terminally to the first polypeptide, N-terminally to the second polypeptide of C-terminally to the second polypeptide. The one or more further polypeptides may be linked to the first or second polypeptide of the chimeric or fusion protein, preferably via a linker of no more than 50 amino acids, or directly linked to the first polypeptide.

In any embodiment, the first polypeptide comprises or consists of an amino acid sequence selected from the group of: SEQ ID NOs: 1 to 11, or sequences at 80%, 81%, 82%, 83%, 84%, 85%, 86%, 87%, 88%, 90%, 91%, 92%, 93%, 94%, 95%, 96%, 97%, 98%, or 99% identical thereto.

The one or more further polypeptides preferably comprise or consist of an amino acid sequence selected from the group of: SEQ ID NOs: 1 to 11, or sequences at least 80%, 81%, 82%, 83%, 84%, 85%, 86%, 87%, 88%, 90%, 91%, 92%, 93%, 94%, 95%, 96%, 97%, 98%, or 99% identical thereto.

In any embodiment, the first polypeptide and the further polypeptide that comprise or consist of an amino acid sequence of the active site of an Arg- or Lys-gingipain of P. gingivalis, comprise or consist of an amino acid sequence that is at least 80%, 81%, 82%, 83%, 84%, 85%, 86%, 87%, 88%, 90%, 91%, 92%, 93%, 94%, 95%, 96%, 97%, 98%, or 99% identical, or wherein the amino acid sequences are identical. The first polypeptide and the further polypeptide may be derived from the active site of heterologous gingipains (e.g., from gingipains of different strains of P. gingivalis). The first polypeptide and the further polypeptide may have an amino acid sequence that is derived from different gingipains (e.g., wherein one of the polypeptides has an amino acid sequence of an active site from a Kgp and the other polypeptide has an amino acid sequence of an active site from an Rgp; or alternatively wherein one of the polypeptides has an amino acid sequence of an active site from a RgpA and the other polypeptide has an amino acid sequence of an active site from an RgpB).

In particularly preferred embodiments of the second aspect of the invention, the chimeric or fusion protein comprises or consists of an amino acid sequence as set forth in any one of SEQ ID NOs: 29, 30, 31, 32, 33, 34, or sequences at least 80%, 81%, 82%, 83%, 84%, 85%, 86%, 87%, 88%, 90%, 91%, 92%, 93%, 94%, 95%, 96%, 97%, 98%, or 99% thereto.

In any embodiment of any aspect of the invention, the chimeric or fusion protein consists or consists essentially of the sequences of the first and second polypeptides as defined herein. It will be appreciated therefore that the chimeric or fusion proteins comprise an arrangement or configuration of domains that differs to the configuration of those domains in naturally occurring gingipain polyprotein sequences. In other words, the first and second polypeptides and domains therein have a differential spatial configuration to naturally occurring gingipain polyproteins.

In any embodiment of any aspect of the invention, the first and second polypeptides are linked. The first and second polypeptides may be linked directly, via a linker, or via a polypeptide sequence of no more than 100, preferably no more than 50 amino acids. Preferably the first and second polypeptides are directly linked, or linked by no more than about 1, 2, 3, 4, 5, 6, 7, 8, 9, 10, 11, 12, 13, 14, or 15 amino acids. Most preferably, the first and second polypeptides are directly linked.

In any embodiment of any aspect of the invention, the C-terminal residue of the first polypeptide may be linked to the N-terminal residue of the second polypeptide, directly, via a linker, or via a polypeptide sequence of no more than 50 amino acids. Alternatively, the N-terminal residue of the first polypeptide may be linked to the C-terminal residue of the second polypeptide, directly, via a linker, or via a polypeptide sequence of no more than 50 amino acids.

In any aspect of the invention, the DUF2436 domain (if present) and ABM domains derived from an Arg- or Lys-gingipain, may be directly linked or joined via a linker, or via a polypeptide sequence. Preferably, the DUF2436 and ABM domains are linked via a short linker sequence comprising about 1, 2, 3, 4, 5, 6, 7, 8, 9, 10, 11, 12, 13, 14, or 15 amino acids. In a particularly preferred embodiment, the DUF and ABM domains are linked via a short linker sequence of no more than about 1, 2, 3, 4, 5, 6, 7, 8, 9, 10, 11, 12, 13, 14, or 15 amino acids.

In preferred embodiments, the DUF2436 and ABM domains (with representative amino acid sequences of SEQ ID NOs: 24 or 25 and 18, respectively may be linked via a peptide sequence having the amino acid sequence EVEDDSP (SEQ ID NO: 46).

In any embodiment, the further polypeptide may be joined directly or via a linker to the chimeric or fusion protein comprising the first and second polypeptides. In embodiments where the further polypeptide is joined to the C terminal region of the fusion protein via the second polypeptide, preferably the C terminus of the second polypeptide is joined directly to the N terminus of the further polypeptide. (For example, the C terminus of the adhesin domain is preferably joined directly to the N terminus of the active site amino acid sequence).

In any embodiment of any aspect of the invention, the linker region is an amino acid sequence of no more than 15 amino acids, and preferably greater than 2 amino acids. Suitable linkers for use in protein constructs, including those with minimal impact on solubility are known in the art. Useful linkers include glycine-serine (GlySer) linkers, which are well-known in the art and comprise glycine and serine units combined in various orders. Examples include, but are not limited to, (GS), (GSGGS)n (SEQ ID NO: 47), (GGGS)n (SEQ ID NO: 48) and (GGGGS)n (SEQ ID NO: 49), where n is an integer of at least one, typically an integer between 1 and about 10, for example, between 1 and about 8, between 1 and about 6, or between 1 and about 5. Other useful linkers include DSSG (SEQ ID NO: 50), DSSGAS (SEQ ID NO: 51), KLDSSG (SEQ ID NO: 52) or others described herein. In certain embodiments, the linker region may be derived from the native gingigipain polyprotein sequence.

The present invention also provides a nucleic acid encoding a chimeric or fusion protein as defined herein.

Preferably, the nucleic acid has a nucleotide sequence that encodes any one or more of the amino acid sequences define in Table 1 herein.

In any embodiment, such a nucleic acid is included in an expression construct in which the nucleic acid is operably linked to a promoter. Such an expression construct can be in a vector, e.g., a plasmid, or viral vector.

The present invention also provides a cell comprising a nucleic acid, or nucleic acid vector as herein described. Examples of cells of the present invention include bacterial cells, yeast cells, insect cells or mammalian cells. Preferably, the cell is isolated, substantially purified or recombinant.

The present invention also provides a composition comprising a chimeric or fusion protein as described herein, optionally in combination with a pharmaceutically acceptable carrier.

The composition may also comprise an adjuvant for potentiating an immune response to the chimeric or fusion protein.

The present invention accordingly further provides for a vaccine or immune stimulating composition for inducing an immune response to P. gingivalis in a subject, the composition comprising:
i) an immunogen in the form of a chimeric or fusion protein as described herein, and
ii) an adjuvant, for potentiating the immune response to the immunogen in the subject.

Preferably, the sole immunogen provided in the compositions, vaccines or immune stimulating compositions of the invention, is a chimeric or fusion protein as herein described.

The present invention also provides a method for inducing an immune response in a subject to P. gingivalis, the method comprising administering to a subject in need thereof, a chimeric or fusion protein, vaccine or immune stimulating composition as described herein.

The present invention also provides a method of inducing a humoral immune response to P. gingivalis in a subject, the method comprising administering to the subject, a chimeric or fusion protein, composition, vaccine or immune stimulating composition as herein defined.

Preferably the immune response that is induced comprises a switch from a Th1 to a Th2 immune response.

It will be appreciated that in any embodiment, the immune response elicited by administration of a chimeric or fusion protein described herein (or vaccine or other composition comprising the same), is preferably antigen-specific. Accordingly, in preferred embodiments, the methods and compositions and chimeric proteins described herein, are for inducing an immune response, preferably a protective immune response, to P. gingivalis gingipain antigens.

In any embodiment, the compositions, chimeric proteins and methods of the invention may be for strengthening an immune response (such as a protective immune response) of a subject to P. gingivalis.

The present invention also provides for methods of immunising a subject against P. gingivalis infection, the method comprising administering to the subject, a chimeric or fusion protein, composition, vaccine or immune stimulating composition as herein defined.

In any embodiment, a subject who has received or has been administered a chimeric or fusion protein of the invention, or composition or vaccine including the same, has an increased level of protection against infection with P. gingivalis, or severity of one or more symptoms of P. gingivalis infection, compared to a subject who has not received the protein, composition or vaccine.

Further still, the invention provides a method of treating a P. gingivalis infection in a subject, the method comprising administering to a subject in need thereof, a chimeric or fusion protein, composition, vaccine or immune stimulating composition as herein defined, thereby treating the P. gingivalis infection in the subject.

The present invention provides a method for reducing or minimising the severity of a symptom associated with an infection with P. gingivalis, comprising administering to an individual in need thereof, a chimeric or fusion protein, composition, vaccine or immune stimulating composition as herein defined, wherein the symptoms are selected from the group consisting of swollen or puffy gums, gums that bleed easily, receding gums, periodontal pockets around the teeth, loss of tooth supporting tissues (periodontal ligament, cementum and/or alveolar bone) pus between gums and teeth, and gingivitis.

The present invention also provides a method for treating P. gingivalis-related disease in a subject, the method comprising administering to an individual in need thereof, a chimeric or fusion protein, composition, vaccine or immune stimulating composition as herein defined. Preferably the P. gingivalis-related disease comprises periodontal disease.

The present invention provides for a method of treatment comprising administration of a chimeric or fusion protein, composition, vaccine or immune stimulating composition of the invention may also comprise administration of one or more of: an antimicrobial compound, an anti-inflammatory agent.

The invention also provides use of a chimeric or fusion protein as herein defined, in the manufacture of a medicament for use in:
inducing an immune response (preferably a protective immune response) in a subject to P. gingivalis;
immunising a subject against P. gingivalis infection;
treating a P. gingivalis infection in a subject;
minimising or reducing the severity of one or more symptoms of P. gingivalis infection; or
treating P. gingivalis-related disease in a subject.

The invention also provides a chimeric or fusion protein, composition, vaccine or immune stimulating composition as herein defined, for use in:
inducing an immune response (preferably a protective immune response) in a subject to P. gingivalis;
immunising a subject against P. gingivalis infection;
treating a P. gingivalis infection in a subject;
minimising or reducing the severity of one or more symptoms of P. gingivalis infection; or
treating P. gingivalis-related disease in a subject.

In any method, use or protein, composition or vaccine for use according to the invention, the subject may be any subject that has or is at risk of having an infection with P. gingivalis. The subject may be a human. The subject may be a veterinary subject, such as a companion animal that has, or is at risk of having an infection with P. gingivalis.

The present invention also provides a method for obtaining an antibody directed to P. gingivalis, the method comprising administering a chimeric or fusion protein, composition, vaccine or immune stimulating composition of the invention, to a non-human animal, thereby generating antibodies directed to P. gingivalis in the animal. Preferably the method further comprises isolating the antibody from the animal (eg, extracting from the blood of the animal) or from an egg thereof (in the case where the animal is an avian species, preferably chicken).

The present invention also provides an antibody preparation comprising an antibody directed to P. gingivalis, wherein the antibody preparation is obtained by administering a chimeric or fusion protein, composition, vaccine or immune stimulating composition of the invention, to a non-human animal, thereby generating antibodies directed to P. gingivalis in the animal, and isolating the antibodies from the animal or egg thereof.

The antibody directed to P. gingivalis may be used therapeutically to eliminate or reduce P. gingivalis infection or prophylactically, to prevent or reduce the severity of P. gingivalis infection.

The present invention also provides a kit comprising a composition comprising a chimeric or fusion protein as herein defined, wherein optionally the kit comprises one or more cytokines and/or adjuvants in sealed containers.

Preferably, the kit comprises a label or package insert indicating that the composition is used for immunising an individual, optionally wherein the label or package insert includes instructions for use.

Throughout this specification, unless the context requires otherwise, the words “comprise,” “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements.

Thus, the phrase “consisting essentially of” indicates that the listed elements are required or mandatory, but that other elements (e.g., 1, 2, 3, 4, 5, 6, 7, 8, 9, 10, 11, 12 13, 14, 15, 16, 17, 18, 19, 20, or more than 20 additional amino acid residues at the N-terminus or C-terminus of a polypeptide sequence) are optional and may or may not be present depending upon whether or not they affect the activity or action of the listed elements.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. A. Mouse periodontitis model—therapeutic vaccination. B. Schematic showing the domain structure of Kgp gingipain polyprotein and components derived therefrom for generating the chimeric or fusion proteins of the invention.

FIG. 2. A. SDS-PAGE of E coli fractions expressing recombinant variants with or without a K1 domain and containing either a full length or truncated DUF domain. Low temperature and low IPTG conditions used. A full length DUF is important for solubility. A full length K1 is detrimental to solubility. B. Native PAGE of chimera structures KDA and KDAK1 in the presence of absence of DTT. Addition of K1 domain to KDA reduces solubility.

FIG. 3. Bone loss study. Statistical analysis—One-way ANOVA and post-hoc Dunnet's T3. #significantly different from the Control Challenge group.

FIG. 4. Antibody isotype response (individual sera) of antigens towards heat-killed P. gingivalis whole cells. Naïve=Control Non Challenge. Gingivalis=Control Challenge with P. gingivalis. Chimera=KDcAK1n antigen. Amox=amoxicillin.

FIG. 5. Bone loss study Statistical analysis—One-way ANOVA and post-hoc Dunnet's T3. #p<0.01; ##p<0.05 (T test) significantly different from the Control Challenge group.

FIG. 6. Bone loss study. S=soluble fraction; Urea=urea used in purification; AC=affinity column purification; IB=inclusion body; Batch=batch purification method. Statistical analysis: one-way ANOVA and post-hoc Dunnet's T3. #p<0.01 significantly different from the Control Challenge group.

FIG. 7. Bone loss study following immunisation of animals with various fusion protein constructs. Statistical analysis: one-way ANOVA and post hoc Dunnet's T3 Significantly different from the Control Challenge group #p<0.05 ##p<0.01 ###p<0.001 ####p<0.0001.

### SEQUENCE INFORMATION

TABLE 1
sequence information

| | SEQ ID | |
|---|---|---|
| Descriptor | No | Sequence |
| KAS (active site | 1 | NTGVSFANYTAHGSETAWADPLLTTSQLKALTNKDK |
| domain) | | |
| Kgp active site | 2 | LNTGV[G/S]FANYTAHGSET[S/A]WADP[S/L][L/V]T[A/T][T/S]Q |
| [432-468] | | [V/L]KALTNK[D/N]K |
| RgpA active | 3 | FNGGISL[V/A]NYTGHGSETAWGTSHFGTTHVKQLTNSNQ |
| site [426-462] | | |
| KAS1 | 4 | LNTGV[G/S]FANYTAHGSET[S/A]WADP[S/L] |
| KAS2 | 5 | NTGV[G/S]FANYTAHGSET[S/A]WADP[S/L][L/V]T[A/T][T/S]Q |
| | | [V/L]KALTNK[D/N]K |
| KAS3 | 6 | V[G/S]FANYTAHGSET[S/A]WADP[S/L][L/V] |
| PAS1K | 7 | LNTGVSFANYTAHGSETAWADP |
| RAS1 | 8 | FNGGISL[V/A]NYTGHGSETAWGTSH |
| RAS2 | 9 | NGGISL[V/A]NYTGHGSETAWGTSHFGTTHVKQLTNSNQ |
| RAS3 | 10 | ISL[V/A]NYTGHGSETAWGTSHF |
| PAS1R | 11 | FNGGISLANYTGHGSETAWGT |
| K1 (CAD | 12 | TLSESFENGIPASWKTIDADGDGHGWKPGNAPGIAGYNSNG |
| domain) | | CVYSESFGLGGIGVLTPDNYLITPALDLPNGGKLTFWVCAQDA |
| | | NYASEHYAVYASSTGNDASNFTNALLEETITAKGVRSPKAIRG |
| | | RIQGTWRQKTVDLPAGTKYVAFRHFQSTDMFYIDLDEVEIKAN |
| | | GKR |
| K1n (truncated | 13 | TLSESFENGIPASWKTIDADGDGHGWKPGNAPGIAGYNSNG |
| CAD domain | | CVYSESFGLGGIGVLTPDNYLITPALDLPNGG |
| K1) | | |
| ABM1 Kgp | 14 | SNEFAPVQNLTGSSVGQKVTLKWDAPNGT |
| [942-970] | | |
| ABM2 Kgp | 15 | ASYTYTVYRDGTKIKEGLTATTFEEDGVAAGNHEYCVEVKYT |
| [885-941] | | AGVSPKVCKDVTVEG |
| ABM2+1 | 16 | ASYTYTVYRDGTKIKEGLTATTFEEDGVAAGNHEYCVEVKYT |
| peptide | | AGVSPKVCKDVTVEGSNEFAPVQNLTGSSVGQKVTLKWDAP |
| [885-970] | | NGT |
| ABM3 Kgp | 17 | PNPNPNPNPNPGT |
| [971-983] | | |
| ABM2+1+3 | 18 | ASYTYTVYRDGTKIKEGLTATTFEEDGVAAGNHEYCVEVKYT |
| peptide | | AGVSPKVCKDVTVEGSNEFAPVQNLTGSSVGQKVTLKWDAP |
| [885-983] | | NGTPNPNPNPNPNPGT |
| ABM 2+3 | 19 | ASYTYTVYRDGTKIKEGLTATTFEEDGVAAGNHEYCVEVKYT |
| | | AGVSPKVCKDVTVEGPNPNPNPNPNPGT |
| ABM1 RgpA | 20 | SNEFAPVQNLTGSAVGQKVTLKWDAPNGT |
| ABM2 RgpA | 21 | SDYTYTVYRDGTKIKEGLTATTFEEDGVA(A/T)GNHEYCVEVK |
| | | YTAGVSPKVCKDVTVEG |
| ABM3 Rgp | 22 | PNPNPNPNPNPNPGT |
| [939-971] | | |
| ABM 2+1+3 | 23 | SDYTYTVYRDGTKIKEGLTATTFEEDGVA(A/T)GNHEYCVEVK |
| Rgp | | YTAGVSPKVCKDVTVEGSNEFAPVQNLTGSAVGQKVTLKWD |
| | | APNGTPNPNPNPNPNPNPGT |
| DUF2436 | 24 | AEGSREVKRIGDGLFVTIEPANDVRANEAKVVLAADNVWGDN |
| | | TGYQFLLDADHNTFGSVIPATGPLFTGTASSNLYSANFEYLIPA |
| | | NADPVVTTQNIIVTGQGEVVIPGGVYDYCITNPEPASGKMWIA |
| | | GDGGNQPARYDDFTFEAGKKYTFTMRRAGMGDGTDM |
| Dc (Truncated | 25 | WGDNTGYQFLLDADHNTFGSVIPATGPLFTGTASSNLYSANF |
| DUF2436) | | EYLIPANADPVVTTQNIIVTGQGEVVIPGGVYDYCITNPEPASG |
| | | KMWIAGDGGNQPARYDDFTFEAGKKYTFTMRRAGMGDGTD |
| | | M |
| DUF2436+ABM | 26 | AEGSREVKRIGDGLFVTIEPANDVRANEAKVVLAADNVWGDN |
| 213 | | TGYQFLLDADHNTFGSVIPATGPLFTGTASSNLYSANFEYLIPA |
| | | NADPVVTTQNIIVTGQGEVVIPGGVYDYCITNPEPASGKMWIA |
| | | GDGGNQPARYDDFTFEAGKKYTFTMRRAGMGDGTDMEVED |
| | | DSPASYTYTVYRDGTKIKEGLTATTFEEDGVAAGNHEYCVEV |
| | | KYTAGVSPKVCKDVTVEGSNEFAPVQNLTGSSVGQKVTLKW |
| | | DAPNGTPNPNPNPNPNPGT |
| DUF2436 + | 27 | AEGSREVKRIGDGLFVTIEPANDVRANEAKVVLAADNVWGDN |
| ABM2+3 | | TGYQFLLDADHNTFGSVIPATGPLFTGTASSNLYSANFEYLIPA |
| | | NADPVVTTQNIIVTGQGEVVIPGGVYDYCITNPEPASGKMWIA |
| | | GDGGNQPARYDDFTFEAGKKYTFTMRRAGMGDGTDMEVED |
| | | DSPASYTYTVYRDGTKIKEGLTATTFEEDGVAAGNHEYCVEV |
| | | KYTAGVSPKVCKDVTVEGPNPNPNPNPNPGT |
| DUF2436 + | 28 | AEGSREVKRIGDGLFVTIEPANDVRANEAKVVLAADNVWGDN |
| ABM2+1 | | TGYQFLLDADHNTFGSVIPATGPLFTGTASSNLYSANFEYLIPA |
| | | NADPVVTTQNIIVTGQGEVVIPGGVYDYCITNPEPASGKMWIA |
| | | GDGGNQPARYDDFTFEAGKKYTFTMRRAGMGDGTDMEVED |
| | | DSPASYTYTVYRDGTKIKEGLTATTFEEDGVAAGNHEYCVEV |
| | | KYTAGVSPKVCKDVTVEGSNEFAPVQNLTGSSVGQKVTLKW |
| | | DAPNGT |
| KDAK | 29 | NTGVSFANYTAHGSETAWADPLLTTSQLKALTNKDKAEGSRE |
| (ie K domain, | | VKRIGDGLFVTIEPANDVRANEAKVVLAADNVWGDNTGYQFL |
| full DUF | | LDADHNTFGSVIPATGPLFTGTASSNLYSANFEYLIPANADPV |
| domain, | | VTTQNIIVTGQGEVVIPGGVYDYCITNPEPASGKMWIAGDGGN |
| ABM2+1+3, K | | QPARYDDFTFEAGKKYTFTMRRAGMGDGTDMEVEDDSPAS |
| domain) | | YTYTVYRDGTKIKEGLTATTFEEDGVAAGNHEYCVEVKYTAG |
| | | VSPKVCKDVTVEGSNEFAPVQNLTGSSVGQKVTLKWDAPNG |
| | | TPNPNPNPNPNPGTNTGVSFANYTAHGSETAWADPLLTTSQL |
| | | KALTNKDK |
| KDA | 30 | NTGVSFANYTAHGSETAWADPLLTTSQLKALTNKDKAEGSRE |
| | | VKRIGDGLFVTIEPANDVRANEAKVVLAADNVWGDNTGYQFL |
| | | LDADHNTFGSVIPATGPLFTGTASSNLYSANFEYLIPANADPV |
| | | VTTQNIIVTGQGEVVIPGGVYDYCITNPEPASGKMWIAGDGGN |
| | | QPARYDDFTFEAGKKYTFTMRRAGMGDGTDMEVEDDSPAS |
| | | YTYTVYRDGTKIKEGLTATTFEEDGVAAGNHEYCVEVKYTAG |
| | | VSPKVCKDVTVEGSNEFAPVQNLTGSSVGQKVTLKWDAPNG |
| | | TPNPNPNPNPNPGT |
| KDAKK | 31 | NTGVSFANYTAHGSETAWADPLLTTSQLKALTNKDKAEGSRE |
| | | VKRIGDGLFVTIEPANDVRANEAKVVLAADNVWGDNTGYQFL |
| | | LDADHNTFGSVIPATGPLFTGTASSNLYSANFEYLIPANADPV |
| | | VTTQNIIVTGQGEVVIPGGVYDYCITNPEPASGKMWIAGDGGN |
| | | QPARYDDFTFEAGKKYTFTMRRAGMGDGTDMEVEDDSPAS |
| | | YTYTVYRDGTKIKEGLTATTFEEDGVAAGNHEYCVEVKYTAG |
| | | VSPKVCKDVTVEGSNEFAPVQNLTGSSVGQKVTLKWDAPNG |
| | | TPNPNPNPNPNPGTNTGVSFANYTAHGSETAWADPLLTTSQL |
| | | KALTNKDKNTGVSFANYTAHGSETAWADPLLTTSQLKALTNK |
| | | DK |
| KDAR | 32 | NTGVSFANYTAHGSETAWADPLLTTSQLKALTNKDKAEGSRE |
| | | VKRIGDGLFVTIEPANDVRANEAKVVLAADNVWGDNTGYQFL |
| | | LDADHNTFGSVIPATGPLFTGTASSNLYSANFEYLIPANADPV |
| | | VTTQNIIVTGQGEVVIPGGVYDYCITNPEPASGKMWIAGDGGN |
| | | QPARYDDFTFEAGKKYTFTMRRAGMGDGTDMEVEDDSPAS |
| | | YTYTVYRDGTKIKEGLTATTFEEDGVAAGNHEYCVEVKYTAG |
| | | VSPKVCKDVTVEGSNEFAPVQNLTGSSVGQKVTLKWDAPNG |
| | | TPNPNPNPNPNPGTNGGISL[V/A]NYTGHGSETAWGTSHFGT |
| | | THVKQLTNSNQ |
| KKDA | 33 | NTGVSFANYTAHGSETAWADPLLTTSQLKALTNKDKKLNTGV |
| | | SFANYTAHGSETAWADPLLTTSQLKALTNKDKAEGSREVKRI |
| | | GDGLFVTIEPANDVRANEAKVVLAADNVWGDNTGYQFLLDAD |
| | | HNTFGSVIPATGPLFTGTASSNLYSANFEYLIPANADPVVTTQ |
| | | NIIVTGQGEVVIPGGVYDYCITNPEPASGKMWIAGDGGNQPA |
| | | RYDDFTFEAGKKYTFTMRRAGMGDGTDMEVEDDSPASYTYT |
| | | VYRDGTKIKEGLTATTFEEDGVAAGNHEYCVEVKYTAGVSPK |
| | | VCKDVTVEGSNEFAPVQNLTGSSVGQKVTLKWDAPNGTPNP |
| | | NPNPNPNPGT |
| KKDAKK | 34 | NTGVSFANYTAHGSETAWADPLLTTSQLKALTNKDKDSSGAS |
| | | NTGVSFANYTAHGSETAWADPLLTTSQLKALTNKDKAEGSRE |
| | | VKRIGDGLFVTIEPANDVRANEAKVVLAADNVWGDNTGYQFL |
| | | LDADHNTFGSVIPATGPLFTGTASSNLYSANFEYLIPANADPV |
| | | VTTQNIIVTGQGEVVIPGGVYDYCITNPEPASGKMWIAGDGGN |
| | | QPARYDDFTFEAGKKYTFTMRRAGMGDGTDMEVEDDSPAS |
| | | YTYTVYRDGTKIKEGLTATTFEEDGVAAGNHEYCVEVKYTAG |
| | | VSPKVCKDVTVEGSNEFAPVQNLTGSSVGQKVTLKWDAPNG |
| | | TPNPNPNPNPNPGTNTGVSFANYTAHGSETAWADPLLTTSQL |
| | | KALTNKDKKLDSSGNTGVSFANYTAHGSETAWADPLLTTSQL |
| | | KALTNKDK |
| KDcAK1n | 35 | NTGVSFANYTAHGSETAWADPLLTTSQLKALTNKDKWGDNT |
| (prior art | | GYQFLLDADHNTFGSVIPATGPLFTGTASSNLYSANFEYLIPA |
| chimera) | | NADPVVTTQNIIVTGQGEVVIPGGVYDYCITNPEPASGKMWIA |
| | | GDGGNQPARYDDFTFEAGKKYTFTMRRAGMGDGTDMEVED |
| | | DSPASYTYTVYRDGTKIKEGLTATTFEEDGVAAGNHEYCVEV |
| | | KYTAGVSPKVCKDVTVEGSNEFAPVQNLTGSSVGQKVTLKW |
| | | DAPNGTPNPNPNPNPNPGTTLSESFENGIPASWKTIDADGDG |
| | | HGWKPGNAPGIAGYNSNGCVYSESFGLGGIGVLTPDNYLITP |
| | | ALDLPNGG |
| KDAK1n | 36 | NTGVSFANYTAHGSETAWADPLLTTSQLKALTNKDKAEGSRE |
| | | VKRIGDGLFVTIEPANDVRANEAKVVLAADNVWGDNTGYQFL |
| | | LDADHNTFGSVIPATGPLFTGTASSNLYSANFEYLIPANADPV |
| | | VTTQNIIVTGQGEVVIPGGVYDYCITNPEPASGKMWIAGDGGN |
| | | QPARYDDFTFEAGKKYTFTMRRAGMGDGTDMEVEDDSPAS |
| | | YTYTVYRDGTKIKEGLTATTFEEDGVAAGNHEYCVEVKYTAG |
| | | VSPKVCKDVTVEGSNEFAPVQNLTGSSVGQKVTLKWDAPNG |
| | | TPNPNPNPNPNPGTTLSESFENGIPASWKTIDADGDGHGWK |
| | | PGNAPGIAGYNSNGCVYSESFGLGGIGVLTPDNYLITPALDLP |
| | | NGG |
| KDAK1 | 37 | NTGVSFANYTAHGSETAWADPLLTTSQLKALTNKDKWGDNT |
| | | GYQFLLDADHNTFGSVIPATGPLFTGTASSNLYSANFEYLIPA |
| | | NADPVVTTQNIIVTGQGEVVIPGGVYDYCITNPEPASGKMWIA |
| | | GDGGNQPARYDDFTFEAGKKYTFTMRRAGMGDGTDMEVED |
| | | DSPASYTYTVYRDGTKIKEGLTATTFEEDGVAAGNHEYCVEV |
| | | KYTAGVSPKVCKDVTVEGSNEFAPVQNLTGSSVGQKVTLKW |
| | | DAPNGTPNPNPNPNPNPGTTLSESFENGIPASWKTIDADGDG |
| | | HGWKPGNAPGIAGYNSNGCVYSESFGLGGIGVLTPDNYLITP |
| | | ALDLPNGGKLTFWVCAQDANYASEHYAVYASSTGNDASNFT |
| | | NALLEETITAKGVRSPKAIRGRIQGTWRQKTVDLPAGTKYVAF |
| | | RHFQSTDMFYIDLDEVEIKANGKR |
| KDcAK1nK | 38 | NTGVSFANYTAHGSETAWADPLLTTSQLKALTNKDKWGDNT |
| Active site | | GYQFLLDADHNTFGSVIPATGPLFTGTASSNLYSANFEYLIPA |
| underlined | | NADPVVTTQNIIVTGQGEVVIPGGVYDYCITNPEPASGKMWIA |
| | | GDGGNQPARYDDFTFEAGKKYTFTMRRAGMGDGTDMEVED |
| | | DSPASYTYTVYRDGTKIKEGLTATTFEEDGVAAGNHEYCVEV |
| | | KYTAGVSPKVCKDVTVEGSNEFAPVQNLTGSSVGQKVTLKW |
| | | DAPNGTPNPNPNPNPNPGTTLSESFENGIPASWKTIDADGDG |
| | | HGWKPGNAPGIAGYNSNGCVYSESFGLGGIGVLTPDNYLITP |
| | | ALDLPNGGNTGVSFANYTAHGSETAWADPLLTTSQLKALTNK |
| | | DK |
| KDAK1nK | 39 | NTGVSFANYTAHGSETAWADPLLTTSQLKALTNKDKAEGSRE |
| Active site | | VKRIGDGLFVTIEPANDVRANEAKVVLAADNVWGDNTGYQFL |
| underlined | | LDADHNTFGSVIPATGPLFTGTASSNLYSANFEYLIPANADPV |
| | | VTTQNIIVTGQGEVVIPGGVYDYCITNPEPASGKMWIAGDGGN |
| | | QPARYDDFTFEAGKKYTFTMRRAGMGDGTDMEVEDDSPAS |
| | | YTYTVYRDGTKIKEGLTATTFEEDGVAAGNHEYCVEVKYTAG |
| | | VSPKVCKDVTVEGSNEFAPVQNLTGSSVGQKVTLKWDAPNG |
| | | TPNPNPNPNPNPGTTLSESFENGIPASWKTIDADGDGHGWK |
| | | PGNAPGIAGYNSNGCVYSESFGLGGIGVLTPDNYLITPALDLP |
| | | NGGNTGVSFANYTAHGSETAWADPLLTTSQLKALTNKDK |
| KKDAK1nKK | 40 | NTGVSFANYTAHGSETAWADPLLTTSQLKALTNKDKDSSGAS |
| K domain | | NTGVSFANYTAHGSETAWADPLLTTSQLKALTNKDKAEGSRE |
| underlined | | VKRIGDGLFVTIEPANDVRANEAKVVLAADNVWGDNTGYQFL |
| Linker in bold | | LDADHNTFGSVIPATGPLFTGTASSNLYSANFEYLIPANADPV |
| | | VTTQNIIVTGQGEVVIPGGVYDYCITNPEPASGKMWIAGDGGN |
| | | QPARYDDFTFEAGKKYTFTMRRAGMGDGTDMEVEDDSPAS |
| | | YTYTVYRDGTKIKEGLTATTFEEDGVAAGNHEYCVEVKYTAG |
| | | VSPKVCKDVTVEGSNEFAPVQNLTGSSVGQKVTLKWDAPNG |
| | | TPNPNPNPNPNPGTTLSESFENGIPASWKTIDADGDGHGWK |
| | | PGNAPGIAGYNSNGCVYSESFGLGGIGVLTPDNYLITPALDLP |
| | | NGGNTGVSFANYTAHGSETAWADPLLTTSQLKALTNKDKKLD |
| | | SSGNTGVSFANYTAHGSETAWADPLLTTSQLKALTNKDK |
| KDcA | 41 | NTGVSFANYTAHGSETAWADPLLTTSQLKALTNKDKWGDNT |
| | | GYQFLLDADHNTFGSVIPATGPLFTGTASSNLYSANFEYLIPA |
| | | NADPVVTTQNIIVTGQGEVVIPGGVYDYCITNPEPASGKMWIA |
| | | GDGGNQPARYDDFTFEAGKKYTFTMRRAGMGDGTDMEVED |
| | | DSPASYTYTVYRDGTKIKEGLTATTFEEDGVAAGNHEYCVEV |
| | | KYTAGVSPKVCKDVTVEGSNEFAPVQNLTGSSVGQKVTLKW |
| | | DAPNGTPNPNPNPNPNPG |
| KDcAK | 42 | NTGVSFANYTAHGSETAWADPLLTTSQLKALTNKDKWGDNT |
| | | GYQFLLDADHNTFGSVIPATGPLFTGTASSNLYSANFEYLIPA |
| | | NADPVVTTQNIIVTGQGEVVIPGGVYDYCITNPEPASGKMWIA |
| | | GDGGNQPARYDDFTFEAGKKYTFTMRRAGMGDGTDMEVED |
| | | DSPASYTYTVYRDGTKIKEGLTATTFEEDGVAAGNHEYCVEV |
| | | KYTAGVSPKVCKDVTVEGSNEFAPVQNLTGSSVGQKVTLKW |
| | | DAPNGTPNPNPNPNPNPGNTGVSFANYTAHGSETAWADPLL |
| | | TTSQLKALTNKDK |
| Dc+2+1+3 | 43 | WGDNTGYQFLLDADHNTFGSVIPATGPLFTGTASSNLYSANF |
| | | EYLIPANADPVVTTQNIIVTGQGEVVIPGGVYDYCITNPEPASG |
| | | KMWIAGDGGNQPARYDDFTFEAGKKYTFTMRRAGMGDGTD |
| | | MEVEDDSPASYTYTVYRDGTKIKEGLTATTFEEDGVAAGNHE |
| | | YCVEVKYTAGVSPKVCKDVTVEGSNEFAPVQNLTGSSVGQK |
| | | VTLKWDAPNGTPNPNPNPNPNPGT |
| Dc+2+3 | 44 | WGDNTGYQFLLDADHNTFGSVIPATGPLFTGTASSNLYSANF |
| | | EYLIPANADPVVTTQNIIVTGQGEVVIPGGVYDYCITNPEPASG |
| | | KMWIAGDGGNQPARYDDFTFEAGKKYTFTMRRAGMGDGTD |
| | | MEVEDDSPASYTYTVYRDGTKIKEGLTATTFEEDGVAAGNHE |
| | | YCVEVKYTAGVSPKVCKDVTVEGPNPNPNPNPNPGT |
| Dc+2+1 | 45 | WGDNTGYQFLLDADHNTFGSVIPATGPLFTGTASSNLYSANF |
| | | EYLIPANADPVVTTQNIIVTGQGEVVIPGGVYDYCITNPEPASG |
| | | KMWIAGDGGNQPARYDDFTFEAGKKYTFTMRRAGMGDGTD |
| | | MEVEDDSPASYTYTVYRDGTKIKEGLTATTFEEDGVAAGNHE |
| | | YCVEVKYTAGVSPKVCKDVTVEGSNEFAPVQNLTGSSVGQK |
| | | VTLKWDAPNGT |

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described. It will be understood that the invention disclosed and defined in this specification extends to all alternative combinations of two or more of the individual features mentioned or evident from the text or drawings. All of these different combinations constitute various alternative aspects of the invention.

All of the patents and publications referred to herein are incorporated by reference in their entirety.

For purposes of interpreting this specification, terms used in the singular will also include the plural and vice versa.

In work leading to the present invention, the inventors investigated various chimeric or fusion proteins for use in inducing immune responses to P. gingivalis and methods for large-scale production of such chimeras for use as vaccine candidates.

The inventors identified various problems associated with large-scale manufacturing and production of chimeric or fusion proteins derived from components of P. gingivalis gingipains. Firstly, the chimeric or fusion proteins described in the prior art are difficult to produce at sufficiently high quantities, as soluble proteins. More specifically, the chimeric proteins of the prior art are typically produced in inclusion bodies in E. coli, making it challenging to produce large amounts of soluble protein for downstream clinical product development.

Reduced solubility when produced in E. coli also substantially contributes to time and scale of manufacturing, as well as poor stability of the proteins once solubilised and refolded from inclusion bodies.

The present invention is therefore concerned with improved design of chimeric or fusion proteins for use in inducing an immune response to P. gingivalis, and methods and uses comprising the same.

In any embodiment, the chimeric or fusion proteins of the present invention have improved solubility and/or stability compared to chimeric or fusion proteins described in the prior art. Improved solubility and/or stability provides for significant advantages when it comes to large-scale production of fusion proteins for use in a clinical setting.

In alternative embodiments, the chimeric or fusion proteins of the invention have a reduced propensity to aggregate compared to chimeric or fusion proteins of the prior art. Aggregation may hinder large-scale manufacturing and production of therapeutic/prophylactic proteins. Consequently, reducing propensity for aggregation of the chimeric proteins of the invention provides for a significant improvement over prior art chimeric proteins for use in inducing immune responses to P. gingivalis.

Further still, in any embodiment, the chimeric or fusion proteins of the invention have increased immunogenicity compared to chimeric or fusion proteins of the prior art.

Thus, the inventors have identified various new approaches for obtaining a chimeric or fusion protein for inducing an immune response to P. gingivalis and which results in a chimeric or fusion protein with one or more advantages over the prior art.

The inventors believe that one or more of the new approaches for generating chimeric or fusion proteins for inducing an immune response to P. gingivalis, as described herein, may result in advantages including: increased ease of manufacturing of a suitable immunogenic composition for inducing an immune response to P. gingivalis; improved shelf-life and/or improved immunogenicity.

### Gingipains

The pathogenicity of P. gingivalis is attributed to a number of surface-associated virulence factors that include cysteine proteinases (gingipains), fimbriae, haem-binding proteins, and outer membrane transport proteins amongst others. In particular, the extracellular Arg- and Lys-specific proteinases ‘gingipains’ (RgpA/B and Kgp) of P. gingivalis have been implicated as major virulence factors that are critical for colonisation, penetration into host tissue, dysregulation of the immune response, dysbiosis and disease.

The gingipains, in particular the Lys-specific proteinase Kgp are essential for P. gingivalis to induce alveolar bone resorption in the mouse periodontitis model. The gingipains have also been found in gingival tissue at sites of severe periodontitis at high concentrations proximal to the subgingival plaque and at lower concentrations at distal sites deeper into the gingival tissue. Lys-specific and Arg-specific proteinases have been shown to degrade a variety of host proteins in vitro, e.g., fibrinogen, fibronectin, and laminin. Plasma host defence and regulatory proteinase inhibitors α-trypsin, α2-macroglobulin, anti-chymotrypsin, antithrombin III and antiplasmin are also degraded by Lys- and Arg-proteinases from P. gingivalis. This has led to the development of a cogent mechanism to explain the keystone role played by P. gingivalis in the development of chronic periodontitis.

The RgpA, RgpB and Kgp genes all encode an N-terminal signal peptide of ~22 amino acids in length, an unusually long propeptide of ~200 amino acids in length, and a catalytic domain of ~480 amino acids. C-terminal to the catalytic domain is a large hemagglutinin-adhesin (HA) domain which is comprised of adhesin binding domains (ABMs, or which 5 distinct sequences have been described), a “Domain of Unknown Function” (termed DUF2436 which is defined as conserved Pfam Domain of Unknown Function; IPR018832) and C-terminal adhesin domains or cleaved adhesin domains (or CADs). The particular arrangement of the ABMs, DUF and CADs varies between Kgp and RgpA/B.

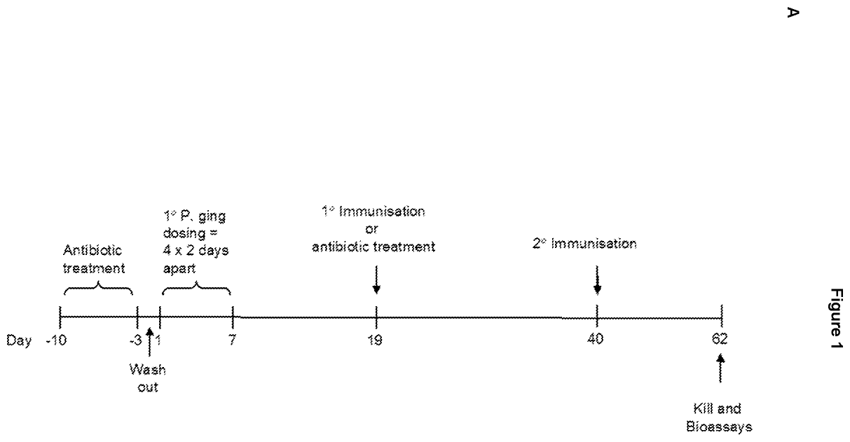

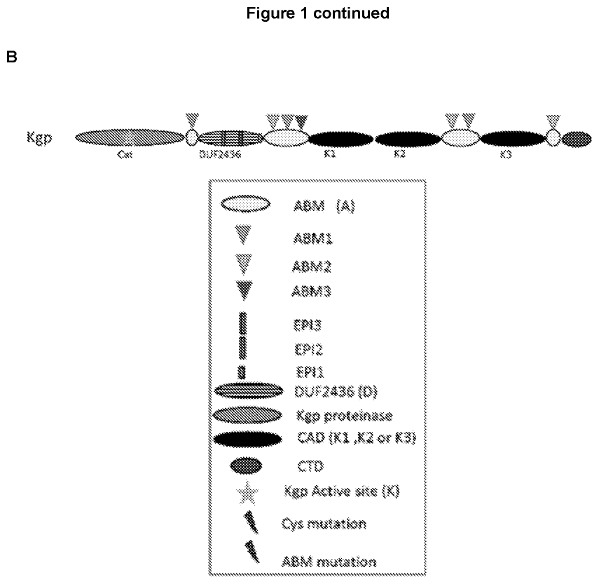

The architecture of the domains in the Kgp polyprotein is illustrated in FIG. 1B. For example, Kgp comprises (N terminus to C terminus): a catalytic domain, a first ABM (ABM1), DUF2436, a domain comprising ABM2, ABM1, ABM3, two CAD domains (termed K1 and K2), a further domain comprising ABM2 and ABM1, a further CAD domain (termed K3), ABM2, and a C-terminal domain.

As used herein, reference to ABMs 1, 2 and 3 will be understood to generally refer to the ABMs found in the order ABM2, ABM1 and ABM3 in the sequence immediately C terminal to DUF2436 of Kgp, as depicted in FIG. 1B.

The catalytic domains of RgpB and RgpA share a high-degree of sequence homology. However, RgpB lacks the HA domains and is located in a monomeric form on the outer membrane. Some of the HA domains have been alternatively described as C-terminal adhesin domains or cleaved adhesin domains (CADs) and some are DUF (“Domain of Unknown Function”) 2436 domains (conserved Pfam Domain of Unknown Function; IPR018832).

The RgpA and Kgp precursor proteins are cleaved into multiple domains that remain non-covalently associated forming large outer membrane protein complexes. In vivo, Arg- and Lys-specific proteinases are therefore found in a cell-associated complex of non-covalently associated proteinases and adhesins. One such complex has been designated the RgpA-Kgp proteinase-adhesin complex (previously referred to as the PrtR-PrtK proteinase-adhesin complex). The complex is composed of a 45 kDa Arg-specific calcium-stabilised cysteine proteinase and seven sequence-related adhesin domains,

As used herein a Lys-gingipain catalytic domain may also be referred to a KAS domain or PAS domain. As used herein an Arg-gingipain catalytic domain may also be referred to as a RAS domain or PAS domain. Typically, the catalytic domain of the Lys-gingipain or Arg-gingipains is located in the N-terminal ~480 amino acid region of the protein. The active site within the catalytic domain is typically located at amino acid residues 426-446 (for RgpA) and 432-453 (for Kgp). Exemplary active site peptides, as found within the catalytic domains are set out in in Table 1 as SEQ ID NOs: 1-11.

As used herein an adhesin domain of an Arg- or Lys-gingipain of P. gingivalis will be understood to typically refer to the region of an Arg- or Lys-gingipain that is C-terminal to the catalytic or active site domain. The adhesin domain (also referred to as the HA domain) typically comprise a Domain of Unknown Function (DUF) domain (especially DUF 2436 conserved Pfam Domain of Unknown Function; IPR018832) and several adhesin binding motifs (ABM) domains and a cleaved adhesin domain (CAD).

### First Polypeptide

The chimeric or fusion proteins of the present invention comprise a first polypeptide that comprises or consists of an amino acid sequence of the active site of an Arg-X or Lys-X proteinase (also referred to herein as Arg- or Lys-gingipain, respectively) of P. gingivalis, or a sequence that is at least 80% identical thereto.

In any embodiment, the first polypeptide comprises or consists of an amino acid sequence selected from the group of: SEQ ID NOs: 1 to 11, or sequences at 80%, 81%, 82%, 83%, 84%, 85%, 86%, 87%, 88%, 90%, 91%, 92%, 93%, 94%, 95%, 96%, 97%, 98%, or 99% identical thereto.

### Improved Immunogenicity

Preferably, the chimeric or fusion protein comprises one or more further polypeptides that comprise or consist of an amino acid sequence of the active site of an Arg- or Lys-gingipain of P. gingivalis, or sequences that are at least 80% identical thereto. The one or more further polypeptides comprising or consisting of the active site of an Arg- or Lys-gingipain of P. gingivalis may be located N-terminally to the first polypeptide, C-terminally to the first polypeptide, N-terminally to the second polypeptide or C-terminally to the second polypeptide. The one or more further polypeptides may be linked to the first or second polypeptide of the chimeric or fusion protein, preferably via a linker of no more than 50 amino acids, or directly linked to the first polypeptide.

The one or more further polypeptides preferably comprise or consist of an amino acid sequence selected from the group of: SEQ ID NOs: 1 to 11, or sequences at least 80%, 81%, 82%, 83%, 84%, 85%, 86%, 87%, 88%, 90%, 91%, 92%, 93%, 94%, 95%, 96%, 97%, 98%, or 99% identical thereto.

In any embodiment, the first polypeptide and the further polypeptide that comprise or consist of an amino acid sequence of the active site of an Arg- or Lys-gingipain of P. gingivalis, comprise or consist of an identical amino acid sequence, or sequences that are at least 80%, 81%, 82%, 83%, 84%, 85%, 86%, 87%, 88%, 90%, 91%, 92%, 93%, 94%, 95%, 96%, 97%, 98%, or 99% identical to each other.

In preferred embodiments, the chimeric or fusion proteins of the invention comprise no more than 2 or nor more than 3 or no more than 4 polypeptides that comprise or consist of an amino acid sequence of the active site of an Arg- or Lys-gingipain of P. gingivalis, or sequences that are at least 80% identical thereto. Preferably, the chimeric or fusion proteins of the invention have fewer than 5, more preferably fewer than 4, most preferably fewer than 3 polypeptides that comprise or consist of an amino acid sequence of the active site of an Arg- or Lys-gingipain of P. gingivalis, or sequences that are at least 80% identical thereto.

### Second Polypeptide

The chimeric or fusion proteins of the present invention comprise a second polypeptide that comprises or consists of an amino acid sequence of an adhesin domain of an ArgX or Lys-X proteinase of P. gingivalis, or a sequence that is at least 80% identical thereto.

It will be understood that the second polypeptide will typically comprise at least a sequence corresponding to one or more of the adhesin-binding motifs (ABM) that are recognised domains/motifs in the adhesin domains of ArgX or Lys-X proteinases of P. gingivalis (such as Kgp and RgpA).

There are five ABMs that have been defined for Kgp and RgpA (ABM1-5). Exemplary sequences for these ABMs are set forth in Table 1.

Typically, the second polypeptide comprises more than one ABM, preferably wherein the second polypeptide comprises at least ABM1 and ABM2, or a sequence at least 80% identical to each of ABM1 or ABM2. The second polypeptide may also comprise the sequence of ABM3 or a sequence at least 80% identical thereto.

It will also be appreciated that the arrangement of the ABM peptides in the second polypeptide need not correspond to the arrangement of the ABM peptides as found in naturally occurring adhesin domains. For example, the ABM peptides may be arranged in the second polypeptide in the sequence (N-terminus to C-terminus) ABM1, ABM2, ABM3 etc. Alternatively, the ABM peptides may be arranged to reflect the arrangement in naturally occurring adhesin domains, such as ABM2, ABM1 and ABM3, such as occurs in Adhesin domain 1 of Kgp.

Further still, the ABMs in the second polypeptide may be arranged contiguously or may be separated from each other by an amino acid sequence of no more than 50 amino acids. It will be appreciated by the skilled person that the spacing between the ABMs is not critical in the design of the chimeric or fusion proteins of the invention.

In any embodiment, the second polypeptide comprises an amino acid sequence as set forth in SEQ ID NO: 16 or a sequence at least 80%, 81%, 82%, 83%, 84%, 85%, 86%, 87%, 88%, 90%, 91%, 92%, 93%, 94%, 95%, 96%, 97%, 98% or 99% identical thereto.

In any embodiment, the second polypeptide comprises an amino acid sequence as set forth in SEQ ID NO: 19 or a sequence at least 80%, 81%, 82%, 83%, 84%, 85%, 86%, 87%, 88%, 90%, 91%, 92%, 93%, 94%, 95%, 96%, 97%, 98% or 99% identical thereto.

In any embodiment, the second polypeptide comprises an amino acid sequence as set forth in SEQ ID NO: 18 or 23 or a sequence at least 80%, 81%, 82%, 83%, 84%, 85%, 86%, 87%, 88%, 90%, 91%, 92%, 93%, 94%, 95%, 96%, 97%, 98% or 99% identical thereto.

In any embodiment, the second polypeptide comprises an amino acid sequence as set forth in SEQ ID NO: 26, 27 or 28, or 43, 44 or 45 or a sequence at least 80%, 81%, 82%, 83%, 84%, 85%, 86%, 87%, 88%, 90%, 91%, 92%, 93%, 94%, 95%, 96%, 97%, 98% or 99% identical thereto.

The inventors have established that the methods of the prior art, which include simply selecting various domains found in naturally occurring gingipains for use in a chimeric or fusion protein typically result in the generation of a protein that is poorly expressed in recombinant systems, and which has a propensity to be expressed as insoluble protein (for example in inclusion bodies in E. coli).

With reference to the examples of the present specification, the inventors recognised that the sequence set forth in SEQ ID NO: 13 (ie, a truncated form of the CAD domain), represents how the Kgp polyprotein is proteolytically processed and assembled on the cell surface. The inclusion of such a sequence was therefore thought to be critical to the induction of an immune response to P. gingivalis. However, the inventors surprisingly found that when present, this sequence contributed to poor solubility and formation of inclusion bodies of recombinant proteins when expressed in E. coli.

The inventors sought to rectify this issue by including the full sequence of the CAD in the fusion proteins. Surprisingly, this did not improve solubility and in fact contributed to lower solubility.

The present inventors have found therefore that the inclusion of a sequence corresponding to one or more cleaved adhesin domains (CADs), or part(s) thereof, contributes to reduced solubility of the proposed chimeric proteins and vaccine compositions derived from gingipain sequences.

As such, in particularly preferred embodiments of the present invention, the second polypeptide does not comprise the sequence set forth in SEQ ID NO: 13, or a sequence at least 80% identical thereto.

Alternatively, the second polypeptide does not comprise a sequence as set forth in SEQ ID NO: 12 or a sequence at least 80% identical thereto or part thereof.

The inventors have also found that the solubility of the chimeric or fusion proteins of the invention is reduced when the chimeric or fusion proteins of the invention comprise only part of the DUF2436 domain (ie an N-terminally truncated DUF2436 domain).

With reference to the examples of the present specification, the inventors recognised prior art approaches to P. gingivalis vaccine design included an N-terminally truncated form of the DUF2436 domain. Although the truncated DUF2436 domain represents how the Kgp polyprotein is proteolytically processed and assembled on the cell surface, the inventors found that providing the sequence of the full length of the DUF2436 domain (ie including the N-terminal portion of the DUF2436 domain) substantially improved production of soluble recombinant protein.

As such, in a preferred embodiment, the second polypeptide in the chimeric or fusion proteins of the invention comprises residues 1 to 37 of a DUF2436 domain, preferably wherein the chimeric protein comprises an amino acid sequence corresponding substantially to the full length of the DUF2436 domain of an Arg- or Lys-gingipain, or a sequence at least 80% identical thereto.

As used herein, an amino acid sequence corresponding substantially to the full length of the DUF2436 domain, or a sequence at least 80% identical thereto, refers to a sequence that is at least 80%, 81%, 82%, 83%, 84%, 85%, 86%, 87%, 88%, 90%, 91%, 92%, 93%, 94%, 95%, 96%, 97%, 98%, 99%, or 100% of the length of a DUF2436 domain of an Arg- or Lys-gingipain.

Preferably the DUF2436 domain comprises the sequence set forth in SEQ ID NO: 24, or sequences at least 90%, 91%, 92%, 93%, 94%, 95%, 96%, 97%, 98% or 99% identical thereto.

### Linking of the First and Second Polypeptides

In the chimeric or fusion proteins of the present invention, the C-terminal residue of the first polypeptide may be covalently linked to the N-terminal residue of the second polypeptide (corresponding to an adhesin domain polypeptide) or the N-terminal residue of the first peptide may be covalently linked to the C-terminal residue of the second polypeptide (corresponding to an adhesin domain polypeptide). In this arrangement, the first peptide and adhesin domain polypeptide, are said to be “directly linked” or “adjacent”.

In other embodiments, the chimeric or fusion protein includes a linker for linking the first peptide to an adhesin domain polypeptide. The linker may be any linker able to join a peptide to a polypeptide, including both amino acid and non-amino acid linkers.

Preferably, the linker is non-immunogenic. Typically, the linker is comprised of amino acids, and may therefore be termed a peptide linker.

A linker is usually a peptide having a length of up to 20 amino acids, although may be longer. The term “linked to” or “fused to” refers to a covalent bond, e.g., a peptide bond, formed between two moieties. Accordingly, in the context of the present invention the linker may have a length of 1, 2, 3, 4, 5, 6, 7, 8, 9, 10, 11, 12, 13, 14, 15, 16, 17, 18, 19, 20, 21 or 22 or more amino acids. For example, the herein provided chimeric or fusion proteins may comprise a linker between the first polypeptide comprising or consisting of an amino acid sequence of a P. gingivalis gingipain active domain, and the second polypeptide corresponding to the adhesin domain of a P. gingivalis gingipain, such as between the N-terminus of the second polypeptide and the C-terminus of the first polypeptide. Such linkers have the advantage that they can make it more likely that the different polypeptides of the fusion protein fold independently and behave as expected. Suitable linkers may be up to 50 amino acids in length, although less than 20, less than 15 or less than five amino acids is preferred. The linker may function to bring the first peptide and adhesin domain polypeptide into a closer spatial arrangement than normally observed in a P. gingivalis trypsin-like enzyme. Alternatively, it may space the first polypeptide and the second polypeptide (corresponding to an adhesin domain polypeptide) apart.

Suitable linkers for use in protein constructs, including those with minimal impact on solubility are known in the art. The linker may be any linker known in the art to the skilled person and may be a flexible linker (such as those comprising repeats of glycine and serine residues), a rigid linker (such as those comprising glutamic acid and lysine residues, flanking alanine repeats) and/or a cleavable linker (such as sequences that are susceptible by protease cleavage). Examples of such linkers are known to the skilled person and are described for example, in Chen et al., (2013) Advanced Drug Delivery Reviews, 65:1357-1369.

Useful linkers include glycine-serine (GlySer) linkers, which are well-known in the art and comprise glycine and serine units combined in various orders. Examples include, but are not limited to, (GS), (GSGGS)n (SEQ ID NO: 47), (GGGS)n (SEQ ID NO: 48) and (GGGGS)n (SEQ ID NO: 49), where n is an integer of at least one, typically an integer between 1 and about 10, for example, between 1 and about 8, between 1 and about 6, or between 1 and about 5.

In some embodiments, the peptide linker may include the amino acids glycine and serine in various lengths and combinations. In some aspects, the peptide linker can include the sequence Gly-Gly-Ser (GGS), Gly-Gly-Gly-Ser (GGGS, SEQ ID NO: 48) or Gly-Gly-Gly-Gly-Ser (GGGGS, SEQ ID NO: 49) and variations or repeats thereof. In some aspects, the peptide linker can include the amino acid sequence GGGGS (a linker of 6 amino acids in length, SEQ ID NO: 49) or even longer. The linker may a series of repeating glycine and serine residues (GS) of different lengths, i.e., (GS)n where n is any number from 1 to 15 or more. For example, the linker may be (GS)3 (i.e., GSGSGS, SEQ ID NO: 53) or longer (GS)₁₁ (SEQ ID NO: 54) or longer. It will be appreciated that n can be any number including 1, 2, 3, 4, 5, 6, 7, 8, 9, 10, 11 or more. Fusion proteins having linkers of such length are included within the scope of the present invention. Similarly, the linker may be a series of repeating glycine residues separated by serine residues. For example (GGGGS)₃ (i.e., the linker may comprise the amino acid sequence GGGGSGGGGSGGGGS, (G4S)3, SEQ ID NO: 55) and variations thereof.

In one embodiment, the peptide linker can include the amino acid sequence GGGGS (a linker of 6 amino acids in length, SEQ ID NO: 49) or even longer. The linker may a series of repeating glycine and serine residues (GS) of different lengths, i.e., (GS)n where n is any number from 1 to 15 or more. For example, the linker may be (GS)3 (i.e., GSGSGS, SEQ ID NO: 53) or longer (GS)₁₁ (SEQ ID NO: 54) or longer. It will be appreciated that n can be any number including 1, 2, 3, 4, 5, 6, 7, 8, 9, 10, 11 or more.

Other useful linkers include DSSG (SEQ ID NO: 50), DSSGAS (SEQ ID NO: 51), KLDSSG (SEQ ID NO: 52) and variations thereof. Examples of other suitable linkers are described in Chen et al., (2013) Advanced Drug Delivery Reviews, 65:1357-1369.

### Chimeric or Fusion Proteins and Recombinant Proteins

The chimeric or fusion proteins of the invention can be prepared by any of a number of conventional techniques although typically, the polypeptides are made using recombinant technology.

In the case of recombinant polypeptides, a DNA fragment encoding a desired peptide can be subcloned into an appropriate vector using well-known molecular genetic techniques (see, e.g., Maniatis et al., Molecular Cloning: A Laboratory Manual, 2nd ed. (Cold Spring Harbor Laboratory, 1982); Sambrook et al., Molecular Cloning A Laboratory Manual, 2nd ed. (Cold Spring Harbor Laboratory, 1989). The fragment can be transcribed and the polypeptide subsequently translated in vitro. Commercially available kits also can be employed (e.g., such as manufactured by Clontech, Palo Alto, Calif.; Amersham Pharmacia Biotech Inc., Piscataway, N.J.; InVitrogen, Carlsbad, Calif., and the like). The polymerase chain reaction optionally can be employed in the manipulation of nucleic acids.

A “fragment” is a portion of a polypeptide of the present invention that retains substantially similar functional activity or substantially the same biological function or activity as the polypeptide, which can be determined using assays described herein.

“Percent (%) amino acid sequence identity” or “percent (%) identical” with respect to a polypeptide sequence, i.e. a polypeptide of the invention defined herein, is defined as the percentage of amino acid residues in a candidate sequence that are identical with the amino acid residues in the specific polypeptide of the invention, after aligning the sequences and introducing gaps, if necessary, to achieve the maximum percent sequence identity, and not considering any conservative substitutions as part of the sequence identity. Reference herein to variants having “at least x % sequence identity” to a recited sequence, means that the variant is at least x % identical to the recited sequence.

In various aspects and embodiments of the invention, the defined polypeptides are described by reference to variants having at least 80% homology to a reference sequence or more. Percentage (%) homology generally refers to a polypeptide of the invention defined herein, defined as the percentage of amino acid residues in a candidate sequence that are identical with the amino acid residues in the specific polypeptide of the invention, after aligning the sequences and introducing gaps, if necessary, to achieve the maximum percent sequence identity, and not considering any conservative substitutions as part of the sequence identity.

The amino acids glycine, alanine, valine, leucine and isoleucine can often be substituted for one another (amino acids having aliphatic side chains). Of these possible substitutions it is preferred that glycine and alanine are used to substitute for one another (since they have relatively short side chains) and that valine, leucine and isoleucine are used to substitute for one another (since they have larger aliphatic side chains which are hydrophobic). Other amino acids which can often be substituted for one another include: phenylalanine, tyrosine and tryptophan (amino acids having aromatic side chains); lysine, arginine and histidine (amino acids having basic side chains); aspartate and glutamate (amino acids having acidic side chains); asparagine and glutamine (amino acids having amide side chains); and cysteine and methionine (amino acids having sulphur containing side chains).

Substitutions of this nature are often referred to as “conservative” or “semi-conservative” amino acid substitutions.

Amino acid deletions or insertions can also be made relative to the native sequence of the P. gingivalis protein. Thus, for example, amino acids which do not have a substantial effect on the activity of the polypeptide, or at least which do not eliminate such activity, can be deleted. Such deletions can be advantageous, particularly with longer polypeptides since the overall length and the molecular weight of a polypeptide can be reduced whilst still retaining activity. This can enable the amount of polypeptide required for a particular purpose to be reduced—for example, dosage levels can be reduced.

Amino acid insertions relative to the sequence of the native polypeptide can also be made. This can be done to alter the properties of a polypeptide for use in the present invention (e.g. to enhance antigenicity).

Amino acid changes can be made using any suitable technique e.g. by using site-directed mutagenesis or solid-state synthesis.

It should be appreciated that amino acid substitutions or insertions within the scope of the present invention can be made using naturally occurring or non-naturally occurring amino acids. Whether or not natural or synthetic amino acids are used, it is preferred that only L-amino acids are present.

Those skilled in the art can determine appropriate parameters for measuring alignment, including any algorithms (non-limiting examples described below) needed to achieve maximal alignment over the full-length of the sequences being compared. When amino acid sequences are aligned, the percent amino acid sequence identity of a given amino acid sequence A to, with, or against a given amino acid sequence B (which can alternatively be phrased as a given amino acid sequence A that has or comprises a certain percent amino acid sequence identity to, with, or against a given amino acid sequence B) can be calculated as: percent amino acid sequence identity=X/Y100, where X is the number of amino acid residues scored as identical matches by the sequence alignment program's or algorithm's alignment of A and B and Y is the total number of amino acid residues in B. If the length of amino acid sequence A is not equal to the length of amino acid sequence B, the percent amino acid sequence identity of A to B will not equal the percent amino acid sequence identity of B to A.

In calculating percent identity, typically exact matches are counted. The determination of percent identity between two sequences can be accomplished using a mathematical algorithm. A nonlimiting example of a mathematical algorithm utilized for the comparison of two sequences is the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the BLASTN and BLASTX programs of Altschul et al. (1990) J. Mol. Biol. 215:403. To obtain gapped alignments for comparison purposes, Gapped BLAST (in BLAST 2.0) can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., BLASTX and BLASTN) can be used. Alignment may also be performed manually by inspection. Another non-limiting example of a mathematical algorithm utilized for the comparison of sequences is the ClustalW algorithm (Higgins et al. (1994) Nucleic Acids Res. 22:4673-4680). ClustalW compares sequences and aligns the entirety of the amino acid or DNA sequence, and thus can provide data about the sequence conservation of the entire amino acid sequence. The ClustalW algorithm is used in several commercially available DNA/amino acid analysis software packages, such as the ALIGNX module of the Vector NTI Program Suite (Invitrogen Corporation, Carlsbad, CA). After alignment of amino acid sequences with ClustalW, the percent amino acid identity can be assessed. A non-limiting examples of a software program useful for analysis of ClustalW alignments is GENEDOC™ or JalView (http://www.jalview.org/). GENEDOC™ allows assessment of amino acid (or DNA) similarity and identity between multiple proteins. Another non-limiting example of a mathematical algorithm utilized for the comparison of sequences is the algorithm of Myers and Miller (1988) CABIOS 4:11-17. Such an algorithm is incorporated into the ALIGN program (version 2.0), which is part of the GCG Wisconsin Genetics Software Package, Version 10 (available from Accelrys, Inc., 9685 Scranton Rd., San Diego, CA, USA). When utilizing the ALIGN program for comparing amino acid sequences, a PAM 120 weight residue table, a gap length penalty of 12, and a gap penalty of 4 can be used.

The polypeptide desirably comprises an amino end and a carboxyl end. The polypeptide can comprise D-amino acids, L-amino acids or a mixture of D- and L-amino acids. The D-form of the amino acids, however, is particularly preferred since a polypeptide comprised of D-amino acids is expected to have a greater retention of its biological activity in vivo.

The term “conservative substitution” as used herein, refers to the replacement of an amino acid present in the native sequence in the peptide with a naturally or non-naturally occurring amino acid or a peptidomimetic having similar steric properties. Where the side-chain of the native amino acid to be replaced is either polar or hydrophobic, the conservative substitution should be with a naturally occurring amino acid, a non-naturally occurring amino acid or with a peptidomimetic moiety which is also polar or hydrophobic (in addition to having the same steric properties as the side-chain of the replaced amino acid).

Conservative amino acid substitution tables providing functionally similar amino acids are well known to one of ordinary skill in the art. The following six groups are examples of amino acids that may be considered to be conservative substitutions for one another:
1) Alanine (A), Serine(S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

As naturally occurring amino acids are typically grouped according to their properties, conservative substitutions by naturally occurring amino acids can be determined bearing in mind the fact that replacement of charged amino acids by sterically similar non-charged amino acids are considered as conservative substitutions. For producing conservative substitutions by non-naturally occurring amino acids it is also possible to use amino acid analogues (synthetic amino acids) well known in the art. A peptidomimetic of the naturally occurring amino acid is well documented in the literature known to the skilled person and non-natural or unnatural amino acids are described further below. When affecting conservative substitutions the substituting amino acid should have the same or a similar functional group in the side chain as the original amino acid.

The phrase “non-conservative substitution” or a “non-conservative residue” as used herein refers to replacement of the amino acid as present in the parent sequence by another naturally or non-naturally occurring amino acid, having different electrochemical and/or steric properties. Thus, the side chain of the substituting amino acid can be significantly larger (or smaller) than the side chain of the native amino acid being substituted and/or can have functional groups with significantly different electronic properties than the amino acid being substituted. Examples of non-conservative substitutions of this type include the substitution of phenylalanine or cyclohexylmethyl glycine for alanine, isoleucine for glycine, or —NH—CH[(—CH2)5-COOH)]—CO— for aspartic acid. Non-conservative substitution includes any mutation that is not considered conservative.

A non-conservative amino acid substitution can result from changes in: (a) the structure of the amino acid backbone in the area of the substitution; (b) the charge or hydrophobicity of the amino acid; or (c) the bulk of an amino acid side chain. Substitutions generally expected to produce the greatest changes in protein properties are those in which: (a) a hydrophilic residue is substituted for (or by) a hydrophobic residue; (b) a proline is substituted for (or by) any other residue; (c) a residue having a bulky side chain, e.g., phenylalanine, is substituted for (or by) one not having a side chain, e.g., glycine; or (d) a residue having an electropositive side chain, e.g., lysyl, arginyl, or histadyl, is substituted for (or by) an electronegative residue, e.g., glutamyl or aspartyl.

Alterations of the native amino acid sequence to produce mutant polypeptides, such as by insertion, deletion and/or substitution, can be done by a variety of means known to those skilled in the art. For instance, site-specific mutations can be introduced by ligating into an expression vector a synthesized oligonucleotide comprising the modified site. Alternately, oligonucleotide-directed site-specific mutagenesis procedures can be used, such as disclosed in Walder et al., Gene 42:133 (1986); Bauer et al., Gene 37:73 (1985); Craik, Biotechniques, 12-19 (January 1995); and U.S. Pat. Nos. 4,518,584 and 4,737,462. A preferred means for introducing mutations is the QuikChange Site-Directed Mutagenesis Kit (Stratagene, LaJolla, Calif.).

Any appropriate expression vector (e.g., as described in Pouwels et al., Cloning Vectors: A Laboratory Manual (Elsevier, N.Y.: 1985)) and corresponding suitable host can be employed for production of recombinant polypeptides. Expression hosts include, but are not limited to, bacterial species within the genera Escherichia, Bacillus, Pseudomonas, Salmonella, mammalian or insect host cell systems including baculovirus systems (e.g., as described by Luckow et al., Bio/Technology 6:47 (1988)), and established cell lines such as the COS-7, C127, 3T3, CHO, HeLa, and BHK cell lines, and the like. The skilled person is aware that the choice of expression host has ramifications for the type of polypeptide produced. For instance, the glycosylation of polypeptides produced in yeast or mammalian cells (e.g., COS-7 cells) will differ from that of polypeptides produced in bacterial cells, such as Escherichia coli.

Alternately, a polypeptide of the invention can be synthesized using standard peptide synthesizing techniques well-known to those of ordinary skill in the art (e.g., as summarized in Bodanszky, Principles of Peptide Synthesis (Springer-Verlag, Heidelberg: 1984)). In particular, the polypeptide can be synthesized using the procedure of solid-phase synthesis (see, e.g., Merrifield, J. Am. Chem. Soc. 85:2149-54 (1963); Barany et al., Int. J. Peptide Protein Res. 30:705-739 (1987); and U.S. Pat. No. 5,424,398). If desired, this can be done using an automated peptide synthesizer. Removal of the t-butyloxycarbonyl (t-BOC) or 9-fluorenylmethyloxycarbonyl (Fmoc) amino acid blocking groups and separation of the polypeptide from the resin can be accomplished by, for example, acid treatment at reduced temperature. The polypeptide-containing mixture can then be extracted, for instance, with dimethyl ether, to remove non-peptidic organic compounds, and the synthesized polypeptide can be extracted from the resin powder (e.g., with about 25% w/v acetic acid). Following the synthesis of the polypeptide, further purification (e.g., using high performance liquid chromatography (HPLC)) optionally can be done in order to eliminate any incomplete polypeptides or free amino acids. Amino acid and/or HPLC analysis can be performed on the synthesized polypeptide to validate its identity. For other applications according to the invention, it may be preferable to produce the polypeptide as part of a larger fusion protein, such as by the methods described herein or other genetic means, or as part of a larger conjugate, such as through physical or chemical conjugation, as known to those of ordinary skill in the art and described herein.

In any embodiment of the invention, the chimeric or fusion protein of the invention may comprise additional amino acid residues to facilitate expression in a recombinant expression system and/or to facilitate purification of the protein. Thus, the proteins defined herein may include additional amino acids such as one, two, three, four, or five amino acids in the N-terminal region. Typically the additional amino acids will include an N-terminal methionine for facilitating expression in recombinant expression systems although it will be appreciated that typically such N-terminal residues are cleaved following translation of the protein. In certain embodiments, the N-terminal amino acids include at least methionine and an alanine residue.

Further, a chimeric or fusion protein according to the invention may include additional amino acids such as one, two, three, four, or five amino acids in the N or C-terminal region, preferably to facilitate purification. It will be understood that such amino acid residues may facilitate the inclusion of a purification tag in the protein (such as histidine tags and the like). Such residues may not be included where untagged versions of the protein are produced.

A polypeptide of the invention may also be modified by, conjugated or fused to another moiety to facilitate purification, or increasing the in vivo half-life of the polypeptides, or for use in immunoassays using methods known in the art. For example, a polypeptide of the invention may be modified by glycosylation, acetylation, pegylation, phosphorylation, amidation, derivatization by known protecting/blocking groups, proteolytic cleavage, linkage to a cellular ligand or other protein, etc.

### Nucleic Acids

Nucleic acid molecules that encode any of the chimeric or fusion proteins or polypeptides of the invention are also within the scope of the invention. The nucleic acids are useful, for example, in making the polypeptides of the present invention and as therapeutic agents. They may be administered to cells in culture or in vivo and may include a secretory signal that directs or facilitates secretion of the polypeptide of the invention from the cell. Also within the scope of the invention are expression vectors and host cells that contain or include nucleic acids of the invention (described further below). While the nucleic acids of the invention may be referred to as “isolated,” by definition, the polypeptides of the invention are not wild-type polypeptides and, as such, would not be encoded by naturally occurring nucleic acids. Thus, while the polypeptides and nucleic acids of the present invention may be “purified,” “substantially purified,” “isolated,” “recombinant” or “synthetic” they need not be so in order to be distinguished from naturally occurring materials.

An “isolated” nucleic acid molecule is a nucleic acid molecule that is identified and separated from at least one contaminant nucleic acid molecule with which it is ordinarily associated in the natural source of the nucleic acid. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes nucleic acid molecules contained in cells that ordinarily express Kgp where, for example, the nucleic acid molecule is in a chromosomal location different from that of natural cells.

The terms “nucleic acid molecule” and “polynucleotide” are used interchangeably herein and refer to a polymeric form of nucleotides of any length, either deoxyribonucleotides or ribonucleotides, or analogues thereof. Non-limiting examples of polynucleotides include a gene, a gene fragment, messenger RNA (mRNA), cDNA, recombinant polynucleotides, plasmids, vectors, isolated DNA of any sequence, isolated RNA of any sequence, nucleic acid probes, and primers. A polynucleotide of the invention may be provided in isolated or purified form. A nucleic acid sequence which “encodes” a selected polypeptide is a nucleic acid molecule which is transcribed (in the case of DNA) and translated (in the case of mRNA) into a polypeptide in vivo when placed under the control of appropriate regulatory sequences. The boundaries of the coding sequence are determined by a start codon at the 5′ (amino) terminus and a translation stop codon at the 3′ (carboxy) terminus. For the purposes of the invention, such nucleic acid sequences can include, but are not limited to, cDNA from viral, prokaryotic or eukaryotic mRNA, genomic sequences from viral or prokaryotic DNA or RNA, and even synthetic DNA sequences. A transcription termination sequence may be located 3′ to the coding sequence.

Polynucleotides of the invention can be synthesised according to methods well known in the art, as described by way of example in Sambrook et al (1989, Molecular Cloning—a laboratory manual; Cold Spring Harbor Press).

The polynucleotide molecules of the present invention may be provided in the form of an expression cassette which includes control sequences operably linked to the inserted sequence, thus allowing for expression of the polypeptide of the invention in vivo in a targeted subject. These expression cassettes, in turn, are typically provided within vectors (e.g., plasmids or recombinant viral vectors) which are suitable for use as reagents for nucleic acid immunization. Such an expression cassette may be administered directly to a host subject. Alternatively, a vector comprising a polynucleotide of the invention may be administered to a host subject. Preferably the polynucleotide is prepared and/or administered using a genetic vector. A suitable vector may be any vector which is capable of carrying a sufficient amount of genetic information and allowing expression of a polypeptide of the invention.

The present invention thus includes expression vectors that comprise such polynucleotide sequences. Thus, the present invention provides a vector for use in preventing or treating an inflammatory disease or condition comprising a polynucleotide sequence which encodes a polypeptide of the invention and optionally one or more further polynucleotide sequences which encode different polypeptides as defined herein.

Furthermore, it will be appreciated that the compositions and products of the invention may comprise a mixture of polypeptides and polynucleotides. Accordingly, the invention provides a composition or product as defined herein, wherein in place of any one of the polypeptide is a polynucleotide capable of expressing said polypeptide.

Expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for expression of a peptide of the invention. Other suitable vectors would be apparent to persons skilled in the art. By way of further example in this regard we refer to Sambrook et al.

Thus, a polypeptide of the invention may be provided by delivering such a vector to a cell and allowing transcription from the vector to occur. Preferably, a polynucleotide of the invention or for use in the invention in a vector is operably linked to a control sequence which is capable of providing for the expression of the coding sequence by the host cell, i.e. the vector is an expression vector.

“Operably linked” refers to an arrangement of elements wherein the components so described are configured so as to perform their usual function. Thus, a given regulatory sequence, such as a promoter, operably linked to a nucleic acid sequence is capable of effecting the expression of that sequence when the proper enzymes are present. The promoter need not be contiguous with the sequence, so long as it functions to direct the expression thereof. Thus, for example, intervening untranslated yet transcribed sequences can be present between the promoter sequence and the nucleic acid sequence and the promoter sequence can still be considered “operably linked” to the coding sequence.

A number of expression systems have been described in the art, each of which typically consists of a vector containing a gene or nucleotide sequence of interest operably linked to expression control sequences. These control sequences include transcriptional promoter sequences and transcriptional start and termination sequences. The vectors of the invention may be for example, plasmid, virus or phage vectors provided with an origin of replication, optionally a promoter for the expression of the said polynucleotide and optionally a regulator of the promoter. A “plasmid” is a vector in the form of an extra-chromosomal genetic element. The vectors may contain one or more selectable marker genes, for example an ampicillin resistance gene in the case of a bacterial plasmid or a resistance gene for a fungal vector. Vectors may be used in vitro, for example for the production of DNA or RNA or used to transfect or transform a host cell, for example, a mammalian host cell. The vectors may also be adapted to be used in vivo, for example to allow in vivo expression of the polypeptide.

A “promoter” is a nucleotide sequence which initiates and regulates transcription of a polypeptide-encoding polynucleotide. Promoters can include inducible promoters (where expression of a polynucleotide sequence operably linked to the promoter is induced by an analyte, cofactor, regulatory protein, etc.), repressible promoters (where expression of a polynucleotide sequence operably linked to the promoter is repressed by an analyte, cofactor, regulatory protein, etc.), and constitutive promoters. It is intended that the term “promoter” or “control element” includes full-length promoter regions and functional (e.g., controls transcription or translation) segments of these regions.

A polynucleotide, expression cassette or vector according to the present invention may additionally comprise a signal peptide sequence. The signal peptide sequence is generally inserted in operable linkage with the promoter such that the signal peptide is expressed and facilitates secretion of a polypeptide encoded by coding sequence also in operable linkage with the promoter.

Typically a signal peptide sequence encodes a peptide of 10 to 30 amino acids for example 15 to 20 amino acids. Often the amino acids are predominantly hydrophobic. In a typical situation, a signal peptide targets a growing polypeptide chain bearing the signal peptide to the endoplasmic reticulum of the expressing cell. The signal peptide is cleaved off in the endoplasmic reticulum, allowing for secretion of the polypeptide via the Golgi apparatus.

### Immunogenic and Vaccine Compositions

The invention further provides compositions comprising the chimeric or fusion proteins defined herein, and the use of such chimeric or fusion proteins in immunogenic or vaccine compositions in the treatment or prevention of P. gingivalis infection.

The term “vaccine composition” used herein is defined as composition used to elicit an immune response against an antigen (immunogen) within the composition in order to protect or treat an organism against disease.

As used herein, the terms “immunostimulating composition”, “vaccine composition” and “immunogenic composition” may generally be used interchangeably.

The immunostimulating compositions or vaccines of the invention may suitably include a pharmaceutically acceptable carrier, excipient, diluent, adjuvant, vehicle, buffer or stabiliser in addition to one or more peptides of the invention as the therapeutically or prophylactically active ingredient. Such carriers include, but are not limited to, saline, buffered saline, dextrose, liposomes, water, glycerol, polyethylene glycol, ethanol and combinations thereof.

The immunostimulating compositions or vaccine compositions can be adapted for administration by any appropriate route, for example by the parenteral (including subcutaneous, intramuscular, intravenous or intradermal or by injection into the cerebrospinal fluid), oral (including buccal or sublingual), nasal, topical (including buccal, sublingual or transdermal), vaginal or rectal route. Such compositions can be prepared by any method known in the art of pharmacy, for example by admixing peptides with the carrier(s) or excipient(s) under sterile conditions. Typically, the vaccine composition is adapted for administration by the subcutaneous, intramuscular, intravenous or intradermal route, typically by injection. Alternatively, the vaccine composition may be adapted for oral or nasal administration.

An immunostimulating composition or vaccine composition adapted for parenteral administration may be an aqueous and non-aqueous sterile injection solution which can contain anti-oxidants, buffers, bacteriostats and solutes which render the formulation substantially isotonic with the blood of the intended recipient; and aqueous and non-aqueous sterile suspensions which can include suspending agents and thickening agents. Excipients which can be used for injectable solutions include water, alcohols, polyols, glycerine and vegetable oils, for example. The composition can be presented in unit-dose or multi-dose containers, for example sealed ampoules and vials, and can be stored in a freeze-dried (lyophilized) condition requiring only the addition of the sterile liquid carried, for example water for injections, immediately prior to use. Extemporaneous injection solutions and suspensions can be prepared from sterile powders, granules and tablets.

An immunostimulating or vaccine composition adapted for oral administration, can be presented as discrete units such as capsules or tablets or lozenges; as powders or granules; as solutions, syrups or suspensions (in aqueous or non-aqueous liquids; or as edible foams or whips; or as emulsions).

Suitable excipients for tablets or hard gelatine capsules include lactose, maize starch or derivatives thereof, stearic acid or salts thereof. Suitable excipients for use with soft gelatine capsules include for example vegetable oils, waxes, fats, semi-solid, or liquid polyols etc.

For the preparation of solutions and syrups, excipients which can be used include for example water, polyols and sugars. For the preparation of suspensions, oils (e.g. vegetable oils) can be used to provide oil-in-water or water in oil suspensions.

An immunostimulating or vaccine composition adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose. A suitable composition wherein the carrier is a liquid, for administration as a nasal spray or as nasal drops, may comprise an aqueous or oil solution of the active ingredient.

Compositions adapted for administration by inhalation include fine particle dusts or mists that can be generated by means of various types of metered dose pressurised aerosols, nebulizers or insufflators.

An immunostimulating or vaccine composition adapted for transdermal administration may be presented as a discrete patch intended to remain in intimate contact with the epidermis of the recipient for a prolonged period of time. For example, the active ingredient can be delivered from the patch by iontophoresis as generally described in Pharmaceutical Research. 3 (6): 318 (1986).

A composition adapted for topical administration may be formulated as an ointment, cream, suspension, lotion, powder, solution (eg mouth wash) paste, gel, spray, aerosol or oil. For infections of the eye or other external tissues, for example mouth and skin, the composition may be applied as a topical ointment or cream. When formulated in an ointment, the active ingredient can be employed with either a paraffinic or a water-miscible ointment base. Alternatively, the active ingredient can be formulated in a cream with an oil-in-water cream base or a water-in-oil base. A pharmaceutical composition adapted for topical administration to the eye may comprise eye drops wherein the active ingredient is dissolved or suspended in a suitable carrier, especially an aqueous solvent. A pharmaceutical composition adapted for topical administration in the mouth may comprise lozenges, pastilles or mouth washes.

The immunostimulating or vaccine composition may contain preserving agents, solubilising agents, stabilising agents, wetting agents, emulsifiers, sweeteners, colourants, odourants, salts (substances of the present invention can themselves be provided in the form of a pharmaceutically acceptable salt), buffers, coating agents or antioxidants.

The vaccine composition of the invention may also contain one or more other prophylactically or therapeutically active agents in addition to the chimeric or fusion protein as defined herein.

A chimeric or fusion protein for use in the vaccine compositions of the invention may or may not be lyophilised.

The vaccine compositions of the invention may also include a pharmaceutically acceptable adjuvant in addition to the peptide(s) as defined herein. Adjuvants are added in order to enhance the immunogenicity of the vaccine composition.

Suitable adjuvants for inclusion in a vaccine composition are known in the art and include incomplete Freund's adjuvant, complete Freund's adjuvant, Freund's adjuvant with MDP (muramyldipeptide), alum (aluminium hydroxide), alum plus Bordatella pertussis and immune stimulatory complexes (ISCOMs, typically a matrix of Quil A containing viral proteins), QS-21, Detox-PC, MPL-SE, MoGM-CSF, TiterMax-G, CRL-1005, GERBU, TERamide, PSC97B, Adjumer, PG-026, GSK-I, GcMAF, B-alethine, MPC-026, Adjuvax, CpG ODN, Betafectin, and MF59.

The vaccine compositions of the invention may also include or be co-administered with one or more co-stimulatory molecules.

Dosages of the vaccine composition of the present invention can vary between wide limits, depending upon the age and condition of the individual to be treated, etc. and a physician will ultimately determine appropriate dosages to be used.

This dosage can be repeated as often as appropriate. For example, an initial dose of the vaccine may be administered and then a booster administered at a later date.

For administration to mammals, and particularly humans, it is expected that the daily dosage of the active agent will be from 1 μg/kg to 10 mg/kg body weight, typically around 10 μg/kg to 1 mg/kg body weight. The physician in any event will determine the actual dosage which will be most suitable for an individual which will be dependent on factors including the age, weight, sex and response of the individual. The above dosages are exemplary of the average case. There can, of course, be instances where higher or lower dosages are merited, and such are within the scope of this invention.

The vaccine composition of the invention can be administered by any convenient route as described herein, such as via the intramuscular, intravenous, by inhalation, intraperitoneal or oral routes or by injection into the cerebrospinal fluid.

The vaccine composition of the invention can be provided in unit dosage form, will generally be provided in a sealed container and may be provided as part of a kit. Such a kit would normally (although not necessarily) include instructions for use. It can include a plurality of said unit dosage forms.

Accordingly, in yet another aspect, the present invention provides a kit of parts comprising a vaccine composition of the invention and one or more cytokines and/or adjuvants in sealed containers.

### Methods for Immunising a Subject Using the Subject Chimeric or Fusion Proteins

The present invention provides methods and compositions for treating or preventing infection or minimising the likelihood of infection with P. gingivalis, in an individual in need thereof, the methods comprising administering a fusion or chimeric protein of the invention.

The present invention also provides for methods and compositions for inducing a a humoural immune response in a subject to P. gingivalis. The humoural response may be for the purposes of obtaining protective/therapeutic anti-P. gingivalis antibodies directly in the individual requiring protection/therapy. Alternatively, the humoural response may be for the purpose of generating antibodies which are then isolated from the subject (or egg thereof), such that the antibodies can then be directly administered to an individual requiring treatment/protection with the antibodies.

As such, the present invention includes methods and compositions for preventing infection with P. gingivalis, minimising the likelihood of infection and/or reducing the severity and duration of P. gingivalis infection in an individual.

The present invention also provides a method for obtaining an antibody directed to P. gingivalis, the method comprising administering a chimeric or fusion protein, composition, vaccine or immune stimulating composition of the invention, to a non-human animal, thereby generating antibodies directed to P. gingivalis in the animal. Preferably the method further comprises isolating the antibody from the animal (eg from the blood of the animal) or from an egg of the animal (eg in the case of generating IgY antibodies from chickens).

The present invention also provides an antibody preparation comprising an antibody directed to P. gingivalis, wherein the antibody preparation is obtained by administering a chimeric or fusion protein, composition, vaccine or immune stimulating composition of the invention, to a non-human animal, thereby generating antibodies directed to P. gingivalis in the animal, and isolating the antibodies from the animal or egg thereof.

The antibody directed to P. gingivalis may be used therapeutically to eliminate or reduce P. gingivalis infection or prophylactically, to prevent or reduce the severity of P. gingivalis infection. In further embodiments, the antibody may be used as a diagnostic or analytical reagent.

As used herein, the terms “treatment” or “treating” of a subject includes the application or administration of a composition of the invention to a subject (or application or administration of a compound of the invention to a cell or tissue from a subject) with the purpose of delaying, slowing, stabilizing, curing, healing, alleviating, relieving, altering, remedying, less worsening, ameliorating, improving, or affecting the disease or condition, the symptom of the disease or condition, or the risk of (or susceptibility to) the disease or condition. The term “treating” refers to any indication of success in the treatment or amelioration of an injury, pathology or condition, including any objective or subjective parameter such as abatement; remission; lessening of the rate of worsening; lessening severity of the disease; stabilization, diminishing of symptoms or making the injury, pathology or condition more tolerable to the subject; slowing in the rate of degeneration or decline; making the final point of degeneration less debilitating; or improving a subject's physical or mental well-being.

As used herein, “preventing” or “prevention” is intended to refer to at least the reduction of likelihood of the risk of (or susceptibility to) acquiring a disease or disorder (i.e., causing at least one of the clinical symptoms of the disease not to develop in a subject that may be exposed to or predisposed to the disease but does not yet experience or display symptoms of the disease). Biological and physiological parameters for identifying such subjects are provided herein and are also well known by physicians.

The vaccine compositions of the invention can be administered to subjects felt to be in greatest need thereof, for example in the context of human patients, to children or the elderly or individuals at risk of exposure to P. gingivalis. The vaccine compositions of the invention can also be administered to subjects suspected of having or diagnosed with having infection with P. gingivalis.

The compositions and methods of the present invention extend equally to uses in both human and/or veterinary medicine, generation of diagnostic agents or the generation of other treatment reagents.

As used herein, the term “subject” shall be taken to mean any animal including humans, for example a mammal. Exemplary subjects include but are not limited to humans and non-human primates. For example, the subject may be a human. In further examples, the subject may be a veterinary subject, such as a companion animal (cat, dog, guinea pig, and the like). As used herein, the terms “subject”, “individual” and “patient” may be used interchangeably.

The skilled person will be familiar with methods for determining successful vaccination/immunisation with a chimeric or fusion protein or composition as described herein. For example, the skilled person will be familiar with methods for quantifying the antibodies generated following immunisation and/or for quantifying the extent of the humoural (Th2) response induced following immunisation.

### Kits

In another embodiment there is provided a kit or article of manufacture including one or more proteins, polypeptides or polynucleotides of the invention and/or immunogenic composition as described above.

In yet another aspect, the present invention provides a kit of parts comprising a vaccine composition of the invention and one or more adjuvants for separate, subsequent or simultaneous administration to a subject.

In other embodiments there is provided a kit for use in a therapeutic or prophylactic application mentioned above, the kit including:
a container holding a protein, polypeptide, polynucleotide or immunogenic composition of the invention;
a label or package insert with instructions for use.

In any embodiment the kit may contain one or more further active principles or ingredients for eliciting an immune response to P. gingivalis in a subject.

The kit or “article of manufacture” may comprise a container and a label or package insert on or associated with the container. Suitable containers include, for example, bottles, vials, syringes, blister pack, etc. The containers may be formed from a variety of materials such as glass or plastic. The container holds a therapeutic composition which is effective for treating the condition and may have a sterile access port (for example the container may be an intravenous solution bag or a vial having a stopper pierceable by a hypodermic injection needle). The label or package insert indicates that the therapeutic composition is used for treating the condition of choice. In one embodiment, the label or package insert includes instructions for use and indicates that the therapeutic or prophylactic composition can be used to treat an inflammatory disease or condition described herein.

The kit may comprise (a) a therapeutic or prophylactic composition; and (b) a second container with a second active principle or ingredient contained therein. The kit in this embodiment of the invention may further comprise a package insert indicating the composition and other active principle can be used to treat a disorder or prevent a complication stemming from an inflammatory disease or condition described herein. Alternatively, or additionally, the kit may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In any embodiment the therapeutic composition may be provided in the form of a device, disposable or reusable, including a receptacle for holding the therapeutic, prophylactic or immunogenic composition. In one embodiment, the device is a syringe, autoinjector or nanopatch. The device may hold between 0.1 to 2 mL of the therapeutic or immunogenic composition. The therapeutic or prophylactic composition may be provided in the device in a state that is ready for use or in a state requiring mixing, dissolution or resuspension or addition of further components.

It will be understood that the invention disclosed and defined in this specification extends to all alternative combinations of two or more of the individual features mentioned or evident from the text or drawings. All of these different combinations constitute various alternative aspects of the invention.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

The following examples describe a series of in vitro and in vivo studies relating to the chimeric or fusion proteins of the invention. Example 1 describes the materials and methods used in the studies describes in Examples 2 and 3. Example 2 describes the results of in vitro studies and Example 3 describes the results of in vivo studies.

### Example 1: Materials and Methods

### 1.1 Materials and Methods for In Vitro Studies

### Expression of Recombinant Proteins

Recombinant proteins (i.e., different chimeric or fusion proteins as described herein) were expressed from pET28 vector (or pDUET-1 for rABM1+rABM2 co-expression from one vector) by induction with isopropyl β-D-thiogalactosidase (IPTG) essentially as follows.

Nucleic acids encoding different chimeras and components of the chimeras and fusion proteins (including active site and adhesin domains) were produced by standard PCR or DNA splicing via overlap extension (“SOEing”) PCR using P. gingivalis W50 genomic DNA template and specifically designed oligonucleotide primers. PCR fragments or SOEn PCR fragments were purified and ligated into cloning vectors pGEMTeasy or pBHA and transformed into chemically competent E. coli α-Gold cells (Bioline, New South Wales, Australia).

DNA sequences encoding single additional KAS (i.e. active site) residues containing either a “DSSG” amino acid linker region (SEQ ID NO: 50) or no linker were sequentially added to the recombinant chimera and Kgp adhesin domains, one at a time, essentially as follows: restriction enzyme sites were introduced onto the ends of DNA insert fragments in chimera and adhesin variant motherclones by PCR with oligonucleotide primers containing nucleotides specific for restriction enzymes. Synthetic DNA fragments encoding an individual KAS sequence with corresponding restriction sequence were ligated onto the insert ends of DNA constructs from motherclones and recombinant clones were purified. Subsequently if a second or third additional KAS was added, further restriction sequences were introduced onto the ends of these cloned inserts and additional DNA fragments encoding KAS sequences with a different restriction site were ligated on one at a time. Motherclone constructs with single, dual or quadruple linear KAS encoding sequences were subject to specific restriction digestion and sub-fragments could then be interchanged via ligation to produce the numerous variants (including those exemplified in Table 1).

Residues within ABM1 and ABM2 were mutated using the QuickChange II Site Directed Mutagenesis kit (Stratagene, La Jolla, CA) following the manufacturer's instructions.

The integrity of every insert in the cloning vectors pGEMTeasy and pBH1 was confirmed by DNA sequencing (Applied Genetics and Diagnostics Facility, The University of Melbourne). Mutations were further verified by DNA sequencing of entire inserts. Verified constructs were then subject to restriction digest with select enzymes and the inserts were cloned into the relevant pET expression vector in E. coli α-select chemically competent cells and subsequently into the E. coli expression host, BL21-CodonPlus (DE3) RIPL (Stratagene, Australia).

Investigation into Features of Chimera which Impact on Solubility of Recombinant Proteins; Small Scale

Single colony transformants were used to inoculate 2 mL of Luria Bertani (LB) broth containing 30 μg/ml kanamycin at 37° C. on an orbital shaker overnight. This inoculum was then used to inoculate (1:100) 2 ml of LB containing 30 μg/ml kanamycin. Cell cultures at OD600=0.6-1.0 were induced with 1 mM IPTG at 37° C. for 2 hours. Cell cultures were centrifuged and the pellet was resuspended in 250 μl PBS and briefly sonicated using a CPX750 Ultrasonic processor (Cole Parmer Instrument Company, USA) and centrifuged.

Total lysate, Soluble (supernatant) and Insoluble (Pellet) fractions were analyzed by SDS-PAGE to assess solubility of the recombinant proteins. Recombinant proteins that remained insoluble or exhibited low solubility under these initial pilot expression conditions were expressed under a variety of conditions with lower temperatures (16° C. to 30° C.) and lower IPTG (0.01-0.5 mM) to ascertain the optimal conditions for enhanced solubility. All recombinant protein inductions were scaled up (20-200 mls) under optimal conditions to test for solubility at medium-large scale. Cultures (200 mL) were subjected to IPTG induction at various temperatures (e.g., 5 h at 30° C. or 16 h at 16° C.).

Cells were harvested and resuspended in lysis buffer (20 mM sodium phosphate, 500 mM NaCl, 0.5% v/v Triton X-100, 5 mg/ml DNAsel, 1× proteinase inhibitor cocktail, 1 mg/ml lysozyme, 10 mM imidazole, pH 8). Cell resuspensions were incubated at 4° C. for 1 h and cleared lysates were then centrifuged (8,000 g, 30 min, 4° C.) to collect the supernatant (soluble) and pellet (insoluble) fractions and analysed by SDS- and NATIVE-PAGE.

### In-Gel HIS Staining of Recombinant Proteins

Recombinant proteins subject to SDS PAGE were subject to staining using Invision In Gel His Stain kit (Invitrogen) following manufacturers' instructions. This stain is highly specific for His-tagged proteins. This procedure was used to identify N-terminal degradation of recombinant proteins since all candidates contained a C-terminal His tag.

### Large Scale Purification of Chimera for Study in Animal Models

The purification conditions for each recombinant protein are detailed in the following sections.

### Protein Expression and Cell Lysis

All recombinant proteins identified for use as vaccine candidates for animal models were expressed as recombinant C-terminally His-tagged fusion proteins or as “tagless” proteins, in E. coli BL21 (DE3) as previously described. The cells were grown at 37° C. in LB medium, TB medium or a modified M9 minimal medium supplemented with 50 μg/mL kanamycin. Cultures at OD600=0.8-1.0 were induced with 0.4 mM IPTG at 37° C. or 34° C. for 2-4 hours, 25° C. for 12 hours, or 16° C. for 16-20 hours. After harvest at 8000 g by centrifugation at 4° C., the cells were lysed for 1.5 h on-ice in lysis buffer [0.35 mg/mL lysozyme, 40 μg/mL DNAse I in TBS150 (50 mM Tris·CI, 150 mM NaCl, pH 7.5], plus 1% Triton X-100 and EDTA-free protease inhibitors (Complete ULTRA Tablets, Sigma-Aldrich) unless indicated otherwise. The cell lysate was clarified by centrifugation for 40 min at 23,500 g at 4° C.

Initiator Met residue (aa 1) on all recombinant proteins denoted in Table 1, beginning with the translated sequence MA is removed in vivo by E. coli N terminal Methionine processing enzymes that cleave Methionine when the penultimate residue is a small residue. This was confirmed by mass spectrometric analyses of KDAK and KDAK-variant recombinant proteins.

Purification of His-Tagged Proteins from Soluble Fractions Under Non-Denaturing Conditions

### 1. Ni-Affinity Chromatography

Cell lysates were clarified by centrifugation at 20,000 g at 4° C. to remove the cell debris. After filtration through a 0.22 μm filter, lysates were loaded to a His Trap Ni-affinity column (GE Healthcare) in loading buffer TBS300 (50 mM Tris·Cl, 300 mM NaCl, pH 7.5), plus 10 mM imidazole and 1% Triton X-100. Columns were washed extensively with the loading buffer and then 20 mM imidazole in TBS300. Bound proteins were eluted with a 20-350 mM imidazole gradient in TBS300 with the absorbance being monitored at 280 nm. Peak fractions were analysed with SDS- and/or native PAGE. Eluted target proteins were concentrated using the Amicon filter units with a molecular weight cutoff of 3 kDa or 10 kDa depending on the protein sizes. The resulting protein solutions were stored at −70° C. for further purification.

### 2. Anion Exchange Chromatography

The isoelectric points (pls) of all candidate proteins were predicted to be in the acidic range using online analysing ProtParam tool-ExPASy (https://web.expasy.org/protparam/), thus anion exchange chromatography was applied for further purification when necessary. Briefly, the concentrated proteins from Ni-affinity purification were diluted into buffer A (50 mM Tris·Cl, 20 mM NaCl, pH 7.5) to reduce ionic strength and then loaded to an anion exchange HiTrap Q column (GE Healthcare) in buffer A. The proteins were eluted with a NaCl gradient from 20 to 400 mM in the Tris buffer with the absorbance at 280 nm monitored. Target proteins in the peak fractions were verified with SDS- and/or native PAGE and concentrated using the Amicon filter units of an appropriate molecular weight cut-off. The protein solutions were stored at −70° C. for further purification or buffer exchange using size exclusion chromatography or dialysis.

### 3. Size Exclusion Chromatography

Size exclusion chromatography was performed on a HiLoad Superdex 200 or a HiLoad Superdex 75 column (GE Healthcare) at 4° C. The proteins from Ni affinity purification or the further step of anion exchange chromatography were loaded to an appropriate column and eluted in the buffer of TBS150 or TBS100 (100 mM NaCl in 50 mM Tris·Cl, pH 7.5) with absorbance at 280 nm monitored. Target proteins in the peak fractions were verified with SDS- and/or native PAGE. For isolation of single oligomeric species such as a dimer, second or further rounds of size exclusion purification were performed. The fractions containing the best protein purity were pooled and concentrated. For preparation of a mixture of the protein homomultimers, the target protein fractions were simply pooled and concentrated. Protein identities were confirmed by determination of intact protein molar masses using liquid chromatography-electrospray ionisation spectrometry (LC-MS, Agilent) or by sequence analysis using liquid chromatography Orbitrap tandem mass spectrometry (LC-MS/MS, Agilent) where necessary. Protein concentration was quantitated by determination of the absorbance at 280 nm using calculated extinction coefficients before submission for animal model experiments.

Purification of His-Tagged Proteins from Inclusion Bodies

### 1. Under Oxidising Conditions

Following lysis of cells (as described for purification from soluble fractions), the insoluble pellet was washed twice with TBS150. The proteins expressed as inclusion bodies were solubilised with 8 M urea at room temperature on a rolling platform for 1 hour in TBS300 or 20 mM NaPi, 500 mM NaCl, pH 7.4 (PBS500). The protein extracts were centrifuged at 20,000 g and filtered through a 0.22 μm filter unit and then loaded to the HisTrap affinity column in the urea containing buffer TBSU (8 M urea in TBS300 plus 20 mM imidazole) or PBSU (8 M urea, 20 mM NaPi pH 7.8, 500 mM NaCl, plus 20 mM imidazole). The column was extensively washed with the same 8 M urea buffer. A further wash with PBSU at pH 6.5 was applied for the columns with proteins loaded in PBSU. For removal of urea, the target proteins were eluted with a gradient of 20-500 mM imidazole in PBSU. When loose Ni-NTA resin (Thermofisher) was used, the urea extracts were mixed with the resin for 2 hours with gentle stirring in PBSU. After extensive wash with PBSU (pH 7.8 and then pH 6.5) and the bound proteins were eluted off the resin with 500 mM imidazole in the same buffer. The eluted proteins were stepwise dialysed into 6 M, 4 M and then 2 M urea phosphate buffers in a dialysis tube with molecular weight cut-off of 3.5 kDa (Fisher Biotec, Australia). The proteins in the 2 M urea buffer were further dialysed into saline only and kept at 4° C. before submission to animal model experiments or otherwise submitted in 2 M urea buffer without further dialysis.

### 2. Under Reducing Conditions

The proteins were extracted from inclusion bodies with 6 M urea buffer in the presence of 5 mM TCEP and loaded to a HisTrap column in the same buffer. After the column was washed with 4 M urea buffer containing 2 M TCEP, the target proteins were refolded on column with a decreasing concentration gradient of urea from 4 to 0 M in the presence of 2 M TCEP. The proteins were then eluted with an increasing concentration gradient of imidazole from 20-350 mM in TBS300 plus 2 mM TCEP. The eluted proteins were then concentrated for size exclusion chromatography in the reducing buffer (2 mM TCEP in TBS150) the same way as described in the section for purification of the protein dimer or multimers from soluble fractions under nondenaturing conditions. After analysis with SDS- and native PAGE, mass spectrometry and quantitation, the samples were stored at −70° C. in the reducing buffer or submitted for animal model experiments.

Purification of His-Tagged Proteins from Soluble Fractions Under Oxidising and Denaturing Conditions

Cell lysates were clarified by centrifugation at 20,000 g to remove debris and insoluble material, then lysates were immediately denatured by addition of a urea solution to a final concentration of 8 M in PBS500 with stirring at room temperature for 1 hour. The resulting lysates were then centrifuged at 20,000 g at room temperature for 40 min. The target proteins were purified with the same approach using HisTrap Ni-affinity column or loose Ni-NTA resin as described in the section of Purification from inclusion bodies-Under oxidising conditions above. The purified antigens in saline were stored at 4° C. before submission to animal model experiments.

Purification of Tagless Candidates from Soluble Fractions

### Anion Exchange Chromatography

This is the first chromatographic step for purification of the tagless proteins. Like their His-tagged counterparts, pls of the tagless candidates were predicted to also be in the acidic range using online analysing ProtParam tool—ExPASy (https://web.expasy.org/protparam/). Therefore, anion exchange chromatography was applied, and two rounds were performed. In the first round, the clarified cell lysates were filtered through a 0.22 μm filter disc and loaded to an anion exchange HiTrap Q column (GE Healthcare) in Buffer AA (20 mM NaPi, pH 6.5, supplemented with 0.05× protease inhibitor cocktails). After wash with 10 column volumes of Buffer AA, the bound proteins were eluted with a NaCl gradient of 0-100% Buffer BA (1M NaCl in 20 mM NaPi pH 6.5) and the absorbance at 280 nm was monitored. Target proteins in the peak fractions were verified with SDS- and/or native PAGE. In the second round, the best fractions from the first round of anion exchange step were pooled and diluted by 6 folds with Buffer AA and were then re-loaded to the anion exchange HiTrap Q column (GE Healthcare) in the same buffer. Bound proteins were eluted with a NaCl gradient of 0-50% Buffer BA with the absorbance at 280 nm being monitored. Target proteins in the peak fractions were verified with SDS- and/or native PAGE and the best fractions were concentrated using the Amicon filter units of a 10 kDa molecular weight cut-off. The protein solutions were stored at −20° C. for further purification with size exclusion chromatography.

### First-Round Size Exclusion Chromatography

Size exclusion chromatography was performed on a HiLoad Superdex 200 column (GE Healthcare) at room temperature. The proteins from purification via anion exchange chromatography were loaded to the size exclusion column and eluted in the buffer of AS (1M (NH₄)₂SO₄, 50 mM NaPi pH 7.0, supplemented with 0.05× protease inhibitor cocktails) with absorbance at 280 nm monitored. Target proteins in the peak fractions were verified with SDS- and/or native PAGE. The fractions of the best protein purity were pooled for further purification with hydrophobic interaction chromatography.

### Hydrophobic Interaction Chromatography

The pooled fractions of target protein in Buffer AS from the first-round size exclusion chromatography was filtered through 0.22 μm membrane and loaded to a HiTrap Phenyl HP column pre-equilibrated in Buffer AH (1M (NH4)2SO4, 50 mM NaPi pH 7) at 4° C. The bound proteins were eluted with a gradient of decreasing ionic strength from 800 mM to 200 mM (NH4)2SO4 in Buffer BH (50 mM NaPi, pH 7) with absorbance at 280 nm monitored. Target protein in the peak fractions were verified with SDS- and/or native PAGE and the best fractions were pooled and concentrated using the Amicon filter units for the next purification or buffer exchange step of the second-round size exclusion chromatography.

### Second-Round Size Exclusion Chromatography

The concentrated samples from the purification step with hydrophobic interactions were loaded to the size exclusion column as used in first-round size exclusion chromatography and eluted in the buffer of BTS (20 mM Bis-Tris, 150 mM NaCl, pH 6.5). Peak fractions were analysed with SDS- and/or native PAGE. The fractions of best purity were pooled and concentrated. After identification with liquid chromatography-electrospray ionisation mass spectrometry (LC-MS, Agilent) and quantitation, the antigens were aliquoted and stored at −80° C. for future use.

### Protein Identification and Quantitation of Tagless Candidates

In addition to SDS- or native PAGE verification, protein identities were confirmed by determination of intact protein molar masses using LC-MS or by sequence analysis using liquid chromatography Orbitrap tandem mass spectrometry (LC-MS/MS, Agilent) where necessary. Protein concentration was quantitated by determination of the absorbance at 280 nm using calculated extinction coefficients (Table 3). Where buffer exchange was necessary before submission for animal model experiments, Superdex 200 size exclusion column (GE Healthcare) or Zeba™ spin desalting columns (ThermoFisher Scientific) were used.

### 1.2 Materials and Methods for In Vivo Studies

### Culture of Bacteria for Mouse Model of Periodontitis

P. gingivalis strain; W50 (serotype C); was obtained from the culture collection of the Oral Health Cooperative Research Centre, The Melbourne Dental School, University of Melbourne, Australia. P. gingivalis W50 was grown on Horse Blood Agar (HBA) (20 g/L HBA; Oxoid Ltd., Hampshire, UK) supplemented with 10% v/v lysed horse blood (37° C.) in an anaerobic N₂ atmosphere containing 5% CO₂ in a MK3 Anaerobic Workstation (Don Whitley Scientific Ltd., Adelaide, Australia). Colonies were inoculated into starter culture comprised of 20 mL sterilised brain heart infusion (37 g/L BHI; Oxoid Ltd., Hamsphire, UK) medium supplemented with 5 mg/L hemin and 0.5 mg/L cysteine [McKee et al. (1986) Infect Immun 52:349-355] and incubated anaerobically (24 h, 37° C.). Absorbance of batch cultures were monitored at OD650 nm using a spectrophotometer (model 295E, Perkin-Elmer, Germany). Bacterial cells were harvested during late exponential growth by centrifugation (7,000 g, 20 min, 4° C.). Bacterial purity was routinely confirmed by Gram stain [Slots (1982). In: Host-Parasite Interaction in Periodontal Disease, Genco, R. J. and Merganhagan, S. E. (eds). Washington D.C.: American Society for Microbiology. pp. 27-45.].

### Preparation of Heat-Killed Bacteria

P. gingivalis W50 culture was harvested (6,500 g, 4° C.), washed once with phosphate buffered saline (PBS) (0.01 M Na2HPO4, 1.5 mM KH₂PO₄ and 0.15 M NaCl, pH 7.4) then pelleted by centrifugation (7,000 g, 20 min 4° C.). Bacterial cells were resuspended in PBS and heated to 65° C. for 15 minutes. The suspension was centrifuged (7,000 g, 20 min 4° C.) and resuspended in sterile PBS and this was repeated once. After the second wash, the supernatant was discarded and the cell pellet was resuspended in sterile PBS to obtain a cell density of 2×10¹⁰ cells/mL, and protein concentration determined using Biorad Protein Assay Dye Reagent Concentrate (Life Science, NSW, Australia).

### Mouse Periodontitis Model

The mouse periodontitis experiments were modified from Baker et al.'s mode (1994). Arch Oral Biol 39:1035-1040) and performed as described previously by O'Brien-Simpson et al. (2005 J Immuno/175:3980-3989). Mice (female BALB/c; 6-8 weeks old, 10 mice/group), on Day 0 were intra-orally inoculated with P. gingivalis consisting of four doses of P. gingivalis W50 [1×10¹⁰ viable P. gingivalis W50 cells suspended in 20 μL PG buffer (50 mM Tris-HCL, 150 mM NaCl, 10 mM MgSO₄ and 14.3 mM Mercaptoethanol, pH 7.4) containing 2% w/v carboxymethylcellulose (CMC, Sigma, New South Wales, Australia)], given two days apart. The inocula were prepared anaerobically and then immediately applied to the gingival margin of the maxillary molar teeth. The number of viable bacteria in each inoculum was verified by flow cytometry and CFU counts on blood agar. Groups of animals consisted of; P. gingivalis W50 orally inoculated (infected control), a non-bacterial inoculated control, and immunised groups. For the therapeutic vaccination periodontitis model (FIG. 1) mice were immunised on day 19 after the first oral inoculation with 50 μg of vaccine candidate in saline/alum (Alhydrogel; 2% aluminium hydroxide wet gel suspension; Invivogen) via the intraperitoneal route. Mice received a second immunisation (50 μg in saline/alum) on day 40 via the subcutaneous route. On Day 62, mice were bled by cardiac puncture and killed. Maxillae were removed and halved through the midline, with 10 halves used to determine alveolar bone loss. Sera were used to determine the antibody profile using ELISA.

### Measurement of Alveolar Bone Loss in Mouse Maxillae

Maxillae to be examined for bone loss were boiled (1 min) in deionised water, mechanically defleshed, and immersed in 2% w/v potassium hydroxide (16 h, 25° C.). Maxillae were washed twice with deionised water (25° C.), dried (1 h, 37° C.) and stained with 0.5% w/v aqueous methylene blue. Digital images of the buccal side of the maxillae were captured with an Olympus DP12 digital camera mounted on a dissecting microscope, using OLYSIA BioReport software version 3.2 (Olympus Australia Pty Ltd, New South Wales, Australia) to assess horizontal bone loss. Maxillae were oriented so that the buccal and lingual molar cusps were superimposed. Images were captured with a micrometre in frame, so that measurements could be standardised for each image. Horizontal bone loss was defined as the loss occurring in a horizontal plane, perpendicular to the alveolar bone crest that resulted in a reduction of the crest height. The visible area from the cemento-enamel junction (CEJ) to the alveolar bone crest (ABC) for each molar was measured using OLYSIA BioReport software version 3.2 imaging software to give the total visible CEJ-ABC area in mm². P. gingivalis-induced alveolar bone loss in mm² was calculated by subtracting the total visible CEJ-ABC area of the uninoculated (N-C) group from the total visible CEJ-ABC area of each experimental group. Alveolar bone loss measurements were determined twice in a random and blinded protocol. Data are expressed as the mean+/−standard deviation in mm² and were analysed using a one-way ANOVA and Dunnetts T3 post-hoc test.

### Pre-Screening of Vaccine Candidates by Enzyme-Linked Immunosorbent Assay (ELISA)

Several monoclonal antibodies to P. gingivalis were used to pre-screen vaccine candidates to determine whether major domains and epitopes present in the vaccine constructs were accessible to the antibodies, thus allowing the generation of an antibody response in vivo. Antigen to be screened was coated onto flat-bottom polyvinyl microtiter plates (Microtiter; Dynatech Laboratories, McLean, VA, US) in 0.1M PBS (pH 7.4) for 16 h, 4° C. In these experiments the following antibody dilution were used; a dilution of 1/4000 dilution of goat anti-mouse; IgG (M8642) antibody (Sigma, New South Wales, Australia). A 1/4000 dilution of a horseradish peroxidase-conjugated swine anti-goat IgG antibody (M5420; Sigma, New South Wales, Australia) was used to develop ELISA experiment.

### Determination of Subclass Antibody in Sera Using ELISA

ELISAs were performed to evaluate subclass antibody in sera as described in Pathirana et al. (2007). Infect Immun 75:1436-1442) using a solution (1 μg/mL) of either HK W50 cells, domain subunits or epitopes in 0.1M PBS (pH 7.4) to coat wells (16 h, 4° C.) of flat-bottom polyvinyl microtiter plates (Microtiter; Dynatech Laboratories, McLean, VA, US). In these experiments the following antibody dilutions were used; a dilution of 1/4000 dilution of goat anti-mouse; IgG (M8642), IgG1 (M8770), IgG2a (M4434) antibodies (Sigma, New South Wales, Australia). A 1/4000 dilution of a horseradish peroxidase-conjugated swine anti-goat IgG antibody (M5420; Sigma, New South Wales, Australia) was used to develop ELISA experiment. For the epitope ELISAs biotinylated peptides were bound to pre-blocked streptavidin coated flat bottom plates (Pierce; Thermo-Fisher) at 10 μg/mL. Following incubation with sera, the ELISA was developed with 1/4000 goat anti-mouse IgG and 1/4000 horseradish peroxidase-conjugated swine anti-goat IgG antibody. All optical density measurements were conducted on a Wallac VICTOR3 1420 Multilabel counter (Perkin Elmer) at 405 nm.

### Example 2: Results of In Vitro Studies

### Chimera Components Impacting Solubility of Recombinant Protein

One chimera previously reported (in WO 2010/022463) consists of a KAS (K) peptide to which is conjugated an N terminally truncated DUF2436 domain (Dc), an adhesin domain comprising ABMs213 (A) and a C terminally truncated CAD domain (truncated K1 domain, termed K1n). This chimera is termed “KDcAK1n” (SEQ ID NO: 35) and may also be referred to herein as “original chimera” or “chimera” in comparison to the chimeric and fusion proteins of the present invention. The KDcAK1n protein is produced as inclusion bodies in E. coli and is poorly soluble and poorly stable.

The truncated DUF and K1 domains of the A1 adhesin fragment represent how the Kgp polyprotein is naturally proteolytically processed and assembled on the P. gingivalis cell surface during infection. It follows that truncated DUF and K1 domains make obvious candidates for inclusion in a vaccine for generating immune responses to gingipains.

Extensive attempts to produce soluble KDcAK1n were undertaken. Variations in growth media, growth conditions, IPTG concentrations, E. coli expression strains, induction temperatures, cell stage of growth at induction, addition of growth stabilisers, extensive testing of lysis buffers and protein storage buffers were systematically investigated. An enhancement of soluble recombinant protein expression was observed in small scale expression under low temperatures and IPTG induction. At small scale, KDcAK1n produced approximately 30-50% of total recombinant protein expression as soluble protein. Nevertheless, the soluble recombinant was unable to be scaled above 10 ml cultures resulting in inclusion body formation with any attempts to scale up.

### Impact of K1 Domain on Solubility

All full DUF, ABM213, ABM21 and DUF-ABM213 domains of the Kgp polyprotein were expressed as highly soluble and stable recombinant proteins. The full K1 domain alone however was insoluble and expressed only as Inclusion Bodies (IB).

Expression of a KAS motif conjugated to a ABM213 domain (ie, “KA”) or a KAS motif conjugated to both DUF and ABM213 domains (ie “KDA”) produced highly soluble, stable recombinant proteins.

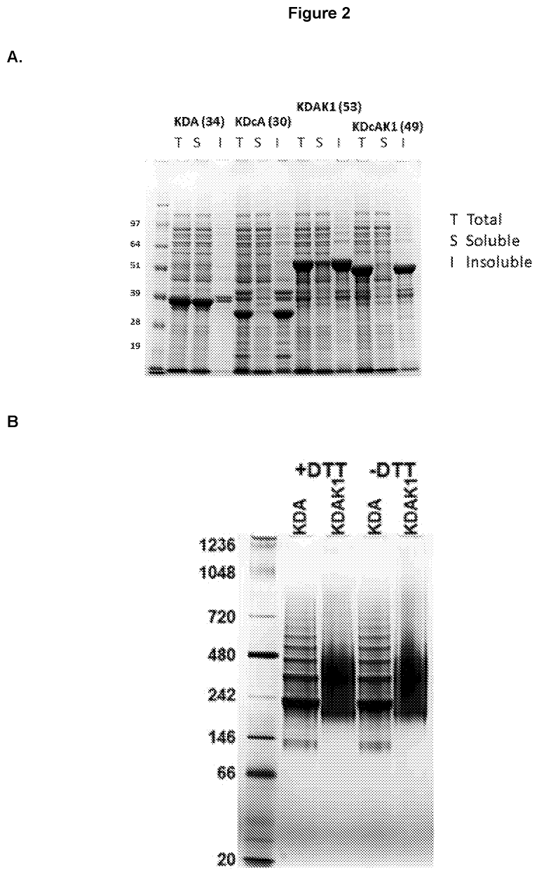

However, as shown in FIG. 2, addition of a C-terminally truncated K1 domain (that is the K1n fragment) or a full K1 domain to recombinants KA or KDA (KAK1 or KDAK1) resulted in compromised solubility.

Both KDAK1n (SEQ ID NO: 36) and KDAK1 (SEQ ID NO: 37) proteins (ie comprising K domain, adhesin domain comprising DUF2436 and ABMs and a truncated or full length CAD domain) require more fastidious growth conditions for soluble expression, at significantly lower levels than for KDA (ie the same protein excluding truncated or full length CAD).

Together with the results for K1 domain expression it is apparent that the K1 (CAD) domain is a contributor to poor solubility of chimera-based recombinants.

### Impact of DUF Domain on Protein Solubility

Truncation of the DUF domain in chimeric protein constructs resulted in elimination of soluble protein expression. This result indicates that a full DUF domain containing the extra 38 N-terminal residues is desirable for optimising soluble expression and efficient stabilization of the recombinant protein.

Thus, a construct expressing a chimera variant with an extended Dc domain (chimera with Dc extended by 38 N terminal residues) was created. This recombinant was designated KDAK1n and results show that chimera solubility is significantly enhanced with a full DUF domain. Expression of KDAK1n in large scale at 30° C. with 0.2-0.5 mM IPTG resulted in expression of a soluble recombinant protein of good yield.

### Impact of K1 and Full Length DUF Domains on Solubility

However, notwithstanding this soluble protein expression at this scale, the inclusion of K1n/K1 in KDAK1n/KDAK1 did appear to make preparation more problematic than that for the KDA variants lacking K1n/K1, such that these variants involving K1 may be less desirable for scale up and commercial development.

To summarise:
A full length DUF domain is desirable for solubility of recombinant variants (FIG. 2A)
The K1 domain (whether full length or truncated) was largely responsible for aggregation and insolubility of variants.
In the absence of a full length DUF domain (ie DUFc), K1 and K1n containing variants were totally insoluble (FIG. 2A).
In the presence of a full length DUF domain, very low-level solubility of K1 and K1n—containing variants was evident under very gentle induction conditions. Nevertheless, this solubility was very low with approximately 80-90% of all recombinant protein being expressed as Inclusion bodies.
NATIVE PAGE gel analysis of mid-scale purifications of soluble KDAK1 recombinants revealed disordered aggregation of recombinant (ie not a neat even ladder as for KDA variants). Small scale purifications of these variants (at low protein concentrations) using mini-Nickle spin columns did not result in similar aggregation however small-scale purification of truncated DUF variants also revealed that characteristic ladders of proteins on NATIVE PAGE were uneven suggesting disordered aggregation.
The K1 and K1n domain containing variants had an increased tendency for aggregation at higher concentrations and after freeze-thaw of purified samples. K1 domain was responsible for this instability. However, all variants lacking K1 or K1n were very stable upon freeze-thaw and at higher concentrations (eg concentrations about 30 mg/ml).

Variants containing a full length or truncated K1 domain were very insoluble and the small level of soluble recombinant protein produced was prone to instability and aggregation after purification. Efficient upscale of soluble K1 containing variants was not possible.

Extension of DUF to full length and/or removal of K1 (or K1n) domains, preferably both, are therefore important for solubility and stability of the recombinant protein.

### Active Site (KAS) Motifs

Additional KAS motifs were engineered onto recombinant chimera variants to determine if this would have an impact on immunogenicity. Single KAS or two successive KAS were added to variants at their N terminus or at both termini. Further, a linear sequence of 4 KAS residues containing DSSG linker sequence (SEQ ID NO: 50) between each KAS motif was also added to the N terminus of selected variants.

Purification profiles and His gel stain analysis of recombinants revealed that additional KAS residues caused some instability and degradation of the recombinant proteins. Degradation and instability was greatest for the 4× linear KAS variants, suggesting that proteolytic processing was occurring within the additional KAS sequences. Subsequent removal of the DSSG linker (SEQ ID NO: 50) between adjacent KAS sequences did not improve stability.

Thus, it was concluded that these variants were prone to proteolytic processing when successive, multiple KAS sequences were expressed in a linear sequence. Soluble variants with a single KAS at one or both termini were relatively stable and less prone to degradation and less prone to compromised purification yields.

### Large Scale Protein Yields and Stability

Most recombinant proteins expressed as soluble proteins and purified under non-denaturing conditions had relatively high yields of their final products (>10 mg/L culture and in excess of 20 mg/L culture).

The yields of these same recombinant proteins when purified from inclusion bodies or from soluble fractions under conditions to deliberately denature them (e.g. urea) were much lower (2.1-3.6 mg/L culture). It was noted that denatured proteins had low binding affinity to the Ni-affinity resin under denaturing conditions and low solubility when finally equilibrated in non-denaturing buffers. In addition, the variants purified from their denatured forms were unstable in unbuffered saline, regardless of whether the protein was initially expressed as a soluble protein or insoluble form and was purified using a prepacked column or using loose Ni-NTA resin. These results suggest that where denaturing agents are not required, it may be desirable to reduce or avoid their usage.

Low expression temperature at 16° C. proved to be effective for improving soluble expression of poorly soluble proteins such as KDAK1 (SEQ ID NO: 37). Nevertheless, enrichment on column in a buffer of low ionic strength during anion exchange purification remarkably compromised yield due to aggregation or precipitation on column, even at the low temperature of 4° C. For these proteins, purification bypassing the anion exchange step resulted in a higher yield, e.g., the yield of KDAK1 was doubled when produced this way. It was also found that KDAK1n (SEQ ID NO: 36) was expressed as a soluble protein, but it did not appear to be stable in solution in later purification process, with a yield of only 2.3 mg/L culture.

Given that K1 is insoluble and that the addition of K1 or K1n to KDA or DA decreases solubility of recombinant variants on large scale purification, as stated above it seems likely that the K1 region may be responsible for instability of K1-containing chimera variants such that exclusion of K1 would be preferred if the domain is not necessary for protection.

Although His staining of gels confirmed that there was degradation within the regions containing multi KAS (K) residues, a negative effect on yield by Ni-affinity chromatography was not observed. For example, the protective antigens, KKDAK1nKK (SEQ ID NO: 40) and KKDAKK (SEQ ID NO: 34) with over 90% of the expected full-length protein species in their final products had yields in a range of 12-21 mg/L culture.

Reducing conditions were found to be favourable for extraction of insoluble KDcAK1n from inclusion bodies using urea. A higher concentration of urea was needed under non-reducing conditions than that under reducing conditions.

The C-term His-tag was not seen to have considerable effects on protein production and solubility when the proteins were expressed under the same conditions.

In purification of tagless candidates, although anion exchange chromatography enriched the target proteins, this step was not able to efficiently separate the target proteins from those of the E. coli host due to close binding affinities to the resin as most E. coli proteins are acidic in a pl range of 4-7 with many of them having close pl to the target proteins. However, interestingly, the size exclusion chromatography at room temperature with the buffer of 1M (NH₄)₂SO₄ appeared to be a critical step and provided a highly efficient separation of target proteins from those of E. coli. After further purification with hydrophobic interaction and the second round of size exclusion chromatography, both lead tagless candidates were purified to a purity of 99% with a high homogeneity, which were evidenced by SDS-/native PAGE and SEC-MALS analyses.

TABLE 2
Summary of recombinant Kgp chimera and fragments expressed in E. coli

| | | (Solubility in 20 ml to 200 ml) | | |
|---|---|---|---|---|
| | | 37 °C, 0.2 to | 16 °C-30 °C, | His Staining |
| Name | Comments | 0.5 mM IPTG | 0.2-0.5 mM IPTG | degradations |
| K1 | CAD domain SEQ ID 12 | (−) | (−) | Not tested |
| DUF | DUF2436 domain | (+++) | | Not tested |
| | (SEQ ID NO: 24) | | | |
| DUF-ABM213 | DUF2436 domain plus | (+++) | | Not tested |
| | ABMs (SEQ ID N): 26) | | | |
| ABM213 | ABM2 + 1 + 3 | (+++) | | Not tested |
| | (SEQ ID NO: 18) | | | |
| ABM21 | SEQ ID NO: 16 | (+++) | | Not tested |
| KDcAK1n | SEQ ID NO: 35 | (−) | (−) | (−) |
| | (truncated DUF, | | | |
| | truncated CAD domain) | | | |
| KDA | KAS domain, Full length | (+++) | (+++) | (+/−) |
| | DUF, ABMs 2 + 1 + 3, | | | |
| | no CAD domain | | | |
| KDAK1n | Full DUF domain and | (+/−) | (+++) | (−/+) |
| | including truncated | | {30 °C} | |
| | CAD domain | | | |
| KDAK1 | Full DUF domain and | (−) | (++) | (+/−) |
| | full length CAD domain | | {16 °C} | |
| KKA | double KAS on | (+++) | | (+++) |
| | N-terminus with | | | |
| | no linker | | | |
| KKDA | double KAS on | (+++) | | (++) |
| | N-terminus with | | | |
| | no linker | | | |
| KKDAK1n | double KAS on | (+/−) | (+++) | (+) |
| | N-terminus with | | {30 °C} | |
| | no linker | | | |
| KDAK | KAS on both termini | (+++) | | (+) |
| | with no linker | | | |
| KDcAK1nK | KAS on both termini | (−) | (−) | (+) |
| | with no linker | | | |
| KDAK1nK | KAS on both termini | (+/−) | (+++) | (+) |
| | with no linker | | {30 °C} | |
| KKAKK | Double KAS on N & C | (+++) | | (+++) |
| | termini with DSSG | | | |
| | linkers | | | |
| KKDAKK | Double KAS on N & C | (+++) | | (++) |
| | termini with DSSG | | | |
| | linkers | | | |
| KKDcAK1nKK | Double KAS on N & C | (−) | (−) | (+) |
| | termini with DSSG | | | |
| | linkers | | | |
| KKDAK1nKK | Double KAS on N & C | (+/−) | (+++) | (+) |
| | termini with DSSG | | {30 °C} | |
| | linkers | | | |
| KKDAK1KK | Double KAS on N & C | (−) | (++) | (+) |
| | termini with DSSG | | {16 °C} | |
| | linkers | | | |
| KKKKA | 4XKAS on N-terminus | (+++) | | (++++) |
| | with DSSG linkers | | | |
| KKKKDA | 4XKAS on N-terminus | (+++) | | (+++) |
| | with DSSG linkers | | | |
| KKKKDcAK1n | 4XKAS on N-terminus | (−) | (−) | (+++) |
| | with DSSG linkers | | | |
| KKKKDAK1n | 4XKAS on N-terminus | (+/−) | (+++) | (+++) |
| | with DSSG linkers | | {30 °C} | |
| KKKKDAK1 | 4XKAS on N-terminus | (−) | (++) | (++) |
| | with DSSG linkers | | {16 °C} | |

### Example 3: Animal Studies

Prevention of P. gingivalis Bone Loss in Mouse Model of Periodontitis

### 1. Experiment 1

Antigens tested: KDcAK1n 50 μg; KDcAK1n 0.5 μg; KDA 50 μg; KDA 0.5 μg; KDAK1 50 μg; KDAK1 0.5 μg. All antigens absorbed onto alum in PBS (pH 7.4).

KDcAK1n: from inclusion bodies, batch purified with Ni-NTA resin followed by dialysis into 2 M urea-PBS under non-reducing conditions.

KDA and KDAK1: from soluble fractions, Ni-affinity (prepacked column) and anion exchange chromatography with gradient elution followed by dialysis into PBS, under non-reducing conditions.

P. gingivalis-Induced Alveolar Bone Loss in Mouse Maxillae

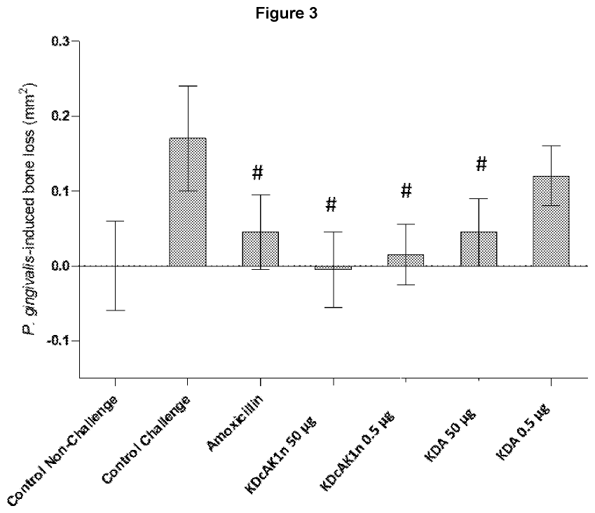

KDcAK1n and KDA protected against bone loss in the animal model. KDcAK1n protected against bone loss at both concentrations tested while KDA only protected at 50 μg (FIG. 3).

### Antibody Response

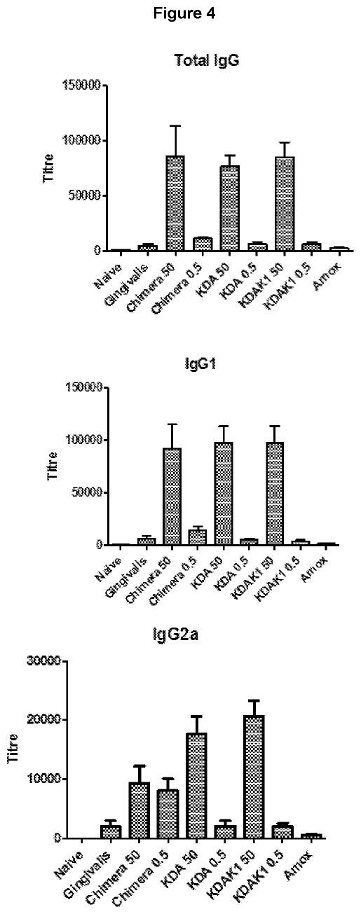

Serum antibody subclass responses of immunised mice in the periodontitis model were examined by ELISA. Antisera was used to probe heat killed P. gingivalis strain W50 as the absorbed antigen. Antibody responses are expressed as the ELISA titre obtained minus triple the background level, with each titre representing the mean±s.d. of the 10 individual mice (FIG. 4). KDcAK1n (referred to in FIG. 4 as “chimera”) and KDA induced the strongest total IgG and IgG1 responses against P. gingivalis whole cells followed by KDAK1. There was no significant difference in IgG and IgG1 antibody responses at 50 μg between the antigens tested.

### 2. Experiment 2

Antigens tested: KDcAK1n 50 μg; KDcAK1n 0.5 μg; KDA 50 μg; KDA 0.5 μg; KDAK1 50 μg; KDAK1 0.5 μg. All antigens absorbed onto alum in saline (pH 7.4).

Antigens were purified in the same way as for Experiment 1.

P. gingivalis-Induced Alveolar Bone Loss in Mouse Maxillae

This experiment examined the same antigens as Experiment 1; however, they were absorbed onto alum using saline rather than PBS. Bone loss results mirrored Experiment 1 and in FIG. 3, (data not shown) where only KDcAK1n and KDA showed protection, indicating using PBS or saline for alum preparation had no effect on the experimental outcome.

### Antibody Response

All antigens tested induced similar total IgG and IgG1 responses against whole cell P. gingivalis. KDA and KDAK1 induced stronger IgG2a responses compared to KDcAK1n (not shown).

### 3. Experiment 3

Antigens tested: KKDA1nKK; KDAK1n; KKDAKK.

All the antigens were purified from soluble fractions under non-reducing conditions with Ni-affinity column, anion exchange and gel filtration chromatography.

### Alum Absorption

The ability of Alhydrogel 2% to absorb antigens was tested by incubating antigens with alum for 60 minutes at 4° C. with gentle mixing. Alum was pelleted and a Bradford protein assay was then conducted on the antigens pre- and post-alum adsorption. All antigens bound to alum at percentages of 91.7% to 99.3%. An SDS-PAGE gel was also run on samples pre- and post-alum absorption and was in agreeance with the Bradford Assay protein determination.

Pre-Screen of Vaccine Candidates with mAbs to P. gingivalis Epitopes

All vaccine candidates were able to bind the KAS2, ABM3 and EP1 mAb, as seen by the large titration curve compared to the negative control muBM4 mAb. KKDA1nKK, KDAK1n and KKDAKK did not bind to ABM2 mAb indicating that the epitope this mAb recognises is not accessible in these constructs (not shown).

P. gingivalis Induced Alveolar Bone Loss in Mouse Maxillae

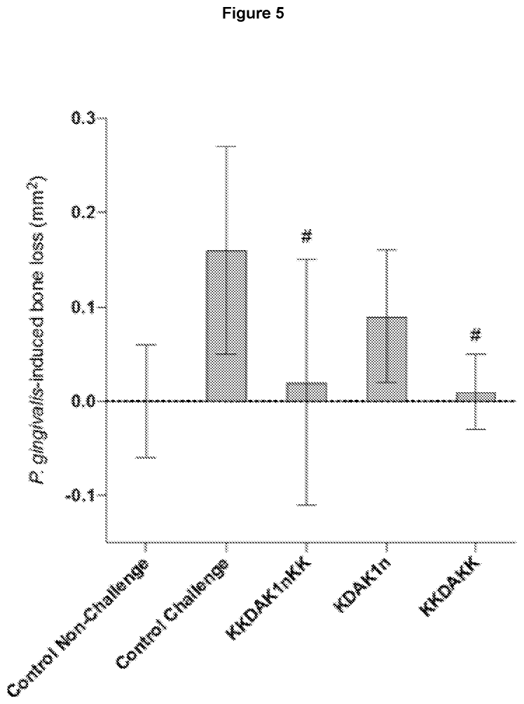

KKDAK1nKK and KKDAKK protected against P. gingivalis-induced bone loss (FIG. 5).

### Antibody Response

Serum antibody subclass responses of immunised mice in the periodontitis model were examined by ELISA. Antisera was used to probe heat killed P. gingivalis strain W50 as the adsorbed antigen. All antigens tested generated an IgG response to varying degrees. The protective KKDAK1nKK and KKDAKK induced robust total IgG and IgG1 responses against heat-killed P. gingivalis.

Pooled serum samples were used to probe P. gingivalis domains adsorbed onto ELISA plates. Total IgG responses against ABM21 (multimer and dimer) and ABM213 (multimer and dimer) were generated by all antigens to varying degrees. An IgG response to DUF2436 was only evident in the antigens that contain the full DUF domain (KKDAK1nKK, KDAK1n, and KKDAKK). Pooled sera samples were also used to probe P. gingivalis epitope peptides and again no clear pattern between protection and non-protection could be observed, although there was a definite tendency for a high KAS titre in the protective antisera.

### 4. Experiment 4

### Antigens Tested

All the antigens were purified under non-reducing conditions using Ni-affinity chromatography followed by dialysis into saline.

KDcAK1n—from inclusion body, using urea and affinity column purified (IB, Urea, AC); KDA—from soluble fraction and affinity column purified (S, AC); KDA—from soluble fraction, using urea and affinity column purified (S, Urea, AC); KDA—from soluble fraction, using urea and batch purification method (S, Urea, Batch); KDAK1—from soluble fraction, using urea and affinity column purified (S, Urea, AC); KDAK1—from inclusion body, using urea and affinity column purified (IB, Urea, AC); KDAK1—from inclusion body, using urea and batch purification method (IB, Urea, Batch).

Pre-Screen of Vaccine Candidates with mAbs to P. gingivalis Epitopes.

All candidates were able to bind the KAS2, ABM3 and EP1 mAb, as seen by the large titration curve compared to the negative control muBM4 mAb. None of the antigens bound the ABM2 mAb, indicating that the epitope this mAb recognises is not accessible in these constructs (not shown).

P. gingivalis Induced Alveolar Bone Loss in Mouse Maxillae

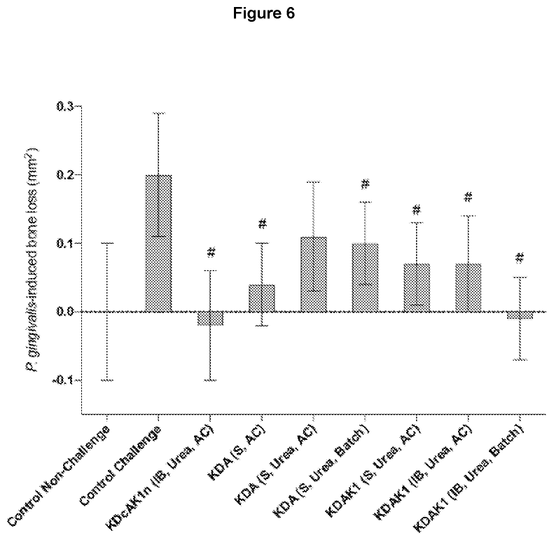

KDcAK1n (IB, Urea, AC), KDA (S, AC), KDAK1 (S, Urea, AC), KDAK1 (IB, Urea, AC) KDAK1 (IB, Urea, Batch) and KDA (S, Urea, Batch) showed significant protection against bone loss with KDcAK1n (IB, Urea, AC), KDA (S, AC), and KDAK1 (IB, Urea, Batch) providing the best protection. It should be noted that the significant protection observed with KDA (S, AC) was lost on denaturation with urea, e.g. KDA (S, Urea, AC) (FIG. 6).

It is noteworthy that unfractionated affinity-purified antigens provided protection of bone loss, in particular good protection was observed with affinity purified soluble KDA but this protection was partially lost on treatment with urea under oxidizing conditions that would promote disulphide cross links of denatured D and A domains. Again these results suggest that urea should be avoided and preparation of soluble, folded domains should be preferred.

### 5. Experiment 5

### Antigens Tested

All the antigens were purified under non-reducing conditions using Ni-affinity chromatography followed by dialysis into saline. Antigens tested were: KDcAK1n—from inclusion body, using urea and affinity column purified (IB, Urea, AC); KDAK1nK, KDAK, KKDAKK, KKDAK1nKK.

P. gingivalis Induced Alveolar Bone Loss in Mouse Maxillae

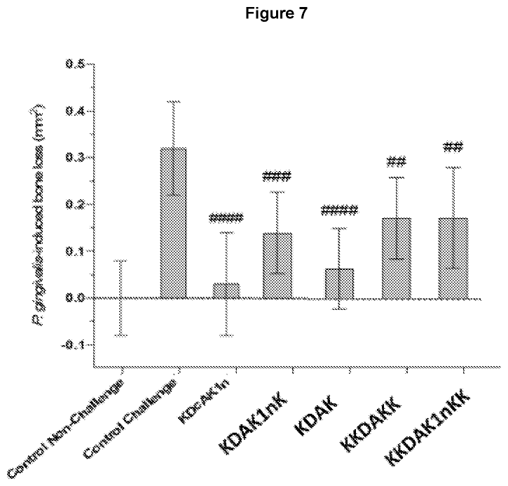

All antigens tested protected against P. gingivalis-induced bone loss. (FIG. 7).

### Summary and Discussion of Results Presented in Examples 2 and 3

The work described in this report compares production and efficacy of various vaccine candidates in order to identify the components of a chimera vaccine that promote ease of production (such as components contributing to improved solubility and stability) and which are most effective in eliciting immune responses to P. gingivalis and/or in reducing P. gingivalis-induced alveolar bone loss.

While efficacious in preventing periodontal bone loss in the animal periodontitis model, a prior art vaccine, KDcAK1n is expressed by E. coli as inclusion bodies and exhibits variable solubility and stability.

KDcAK1n was based on a fusion between the active site sequence (KAS or K) of the Lys-specific gingipain Kgp and the processed adhesin fragment (A1) of the Kgp polyprotein. From structural analysis of the Kgp polyprotein domains it is now clear that the A1 adhesin fragment found on the cell surface comprises three discrete structural domains, the DUF domain (D), the ABM domain (A) and the K1 domain (K1). The processing of the Kgp polyprotein on the surface of P. gingivalis to release the proteinase catalytic domain and adhesins involves N-terminal truncation of the DUF domain by 38 amino acid residues. However, adding these extra N-terminal 38 residues to the construct to produce the full DUF domain instead of the truncated domain (Dc) produced a highly soluble recombinant protein (e.g. DUF2436, KDA and KKDAKK).

KDcAK1n also included a C-terminally truncated K1 domain (K1n) again based on the cell surface processing of the Kgp polyprotein to produce the A1 adhesin fragment. The full K1 domain with the addition of the missing C-terminal sequence to K1n was not improved in terms of solubility as the protein was still expressed largely as inclusion bodies by E. coli. As most constructs with partial or complete K1 domains exhibited some solubility issues it was concluded that the full DUF domain was important for expression of constructs as soluble proteins by E. coli and that inclusion of the K1 domain in the chimera variants should be avoided if possible.

Furthermore, from periodontal bone loss and epitope analysis data from the animal experiments it appeared that the K1 domain may not add significantly to protection, particularly where none of the protective antigens generated anti-sera that bound to the K1 epitope ABM5. Thus, omission of the K1 (or K1n) domain improves solubility, without significantly impacting on vaccine efficacy.

An examination of antibody responses to the different segments of the chimera (K, D, A and K1) and suspected important epitopes (KAS, ABM2, ABM3, EP1) using protective and non-protective sera did not provide any obvious patterns, although it was clear that those chimera variants that protected tended to show strong (IgG/IgG1) responses against whole P. gingivalis cells as well as against the known protective epitopes (KAS, DUF, ABM) such that it appeared that protection may not be related to any one epitope but more to a combination of epitopes being required to ensure full protection of periodontal bone loss.

1-50. (canceled)

51. A chimeric or fusion protein for inducing an immune response to P. gingivalis, the protein comprising a first polypeptide and a second polypeptide, wherein:
A) the first polypeptide comprises or consists of an amino acid sequence of the active site of an Arg- or Lys-gingipain of P. gingivalis, or a sequence that is at least 80% identical thereto; and
B) the second polypeptide comprises or consists of an amino acid sequence of an adhesin domain of an Arg- or Lys-gingipain of P. gingivalis;
wherein the second polypeptide comprises the sequence of one or more adhesin binding motifs (ABMs),
and wherein the second polypeptide:
a) does not comprise: a sequence of a cleaved adhesin domain (CAD), and/or
b) comprises an amino acid sequence corresponding substantially to the full length of the DUF2436 domain of an Arg- or Lys-gingipain, or a sequence at least 80% identical thereto.

52. A chimeric or fusion protein according to claim 51, wherein the one or more ABMs comprise the sequence as set forth in SEQ ID NO: 15 or 21, SEQ ID NO: 14 or 20 and/or SEQ ID NO: 17 or 22 or sequences at least 80% identical thereto, or wherein the one or more ABMs comprise the sequence set forth in any of SEQ ID NO: 16 or SEQ ID NO: 18, 19 or 23 or a sequence at least 80% identical thereto.

53. A chimeric or fusion protein according to claim 51, wherein the chimeric or fusion protein does not comprise a sequence of a CAD, or part thereof, having the amino acid sequence set forth in SEQ ID NO: 12 or 13 or a sequence at least 80% identical thereto.

54. A chimeric or fusion protein according to claim 51, wherein the amino acid sequence corresponding substantially to the full length of the DUF2436 domain of an Arg- or Lys-gingipain is a sequence that is at least 80%, 85%, 90%, 91%, 92%, 93%, 94%, 95%, 96%, 97%, 98%, 99%, or 100% of the length of a DUF2436 domain of an Arg- or Lys-gingipain, or a sequence set forth in SEQ ID NO: 24 or is a sequence at least 80%, 81%, 82%, 83%, 84%, 85%, 86%, 87%, 88%, 90%, 91%, 92%, 93%, 94%, 95%, 96%, 97%, 98%, or 99% identical thereto.

55. A chimeric or fusion protein according to claim 51, wherein the second polypeptide comprises the sequence set forth in any one of SEQ ID NOs: 26, 27, 28, or a sequence at least 80%, 81%, 82%, 83%, 84%, 85%, 86%, 87%, 88%, 90%, 91%, 92%, 93%, 94%, 95%, 96%, 97%, 98%, or 99% identical thereto.

56. A chimeric or fusion protein according to claim 51, wherein the first polypeptide is joined directly to the second polypeptide, or wherein the first polypeptide is linked to the second polypeptide via a linker.

57. A chimeric or fusion protein according to claim 51, wherein the first polypeptide is linked to the second polypeptide via a peptide linker that comprises at least 2 amino acids, at least 5 amino acids, at least 10 amino acids, at least 15 amino acids, at least 20 amino acids, and no more than 50 amino acids.

58. A chimeric or fusion protein according to claim 51, wherein the first polypeptide comprises or consists of the amino acid sequence as set forth in any one of SEQ ID NOs: 1 to 11, or sequences at least 80%, 81%, 82%, 83%, 84%, 85%, 86%, 87%, 88%, 90%, 91%, 92%, 93%, 94%, 95%, 96%, 97%, 98%, or 99% identical thereto.

59. A chimeric or fusion protein according to claim 51, wherein the chimeric or fusion protein comprises one or more further polypeptides that comprise or consist of an amino acid sequence of the active site of an Arg- or Lys-gingipain of P. gingivalis, or sequences that are at least 80% identical thereto.

60. A chimeric or fusion protein according to claim 59, wherein the one or more further polypeptides is located N-terminally to the first polypeptide, C-terminally to the first polypeptide, N-terminally to the second polypeptide or C-terminally to the second polypeptide.

61. A chimeric or fusion protein according to claim 59, wherein the one or more further polypeptides is directly linked to the first or second polypeptide of the chimeric or fusion protein, or wherein the one or more further polypeptides is linked to the first or second polypeptide by a peptide linker.

62. A chimeric or fusion protein according to claim 59, wherein the one or more further polypeptides comprise or consist of an amino acid sequence selected from the group of: SEQ ID NOs: 1 to 11, or sequences at least 80%, 81%, 82%, 83%, 84%, 85%, 86%, 87%, 88%, 90%, 91%, 92%, 93%, 94%, 95%, 96%, 97%, 98%, or 99% identical thereto.

63. A chimeric or fusion protein according to claim 59, wherein the first polypeptide and the one or more further polypeptides comprise the amino acid sequences of the active site of a Lys-gingipain, or wherein the first polypeptide comprises an amino acid sequence of the active site of a Lys-gingipain, and the one or more further polypeptides comprise the amino acid sequence of the active site of an Arg-gingipain.

64. A chimeric or fusion protein according to claim 51, wherein the chimeric or fusion protein comprises or consists of an amino acid sequence as set forth in any one of SEQ ID NOs: 29, 30, 31, 32, 33, 34, 41 or 42 or sequences at least 80%, 81%, 82%, 83%, 84%, 85%, 86%, 87%, 88%, 90%, 91%, 92%, 93%, 94%, 95%, 96%, 97%, 98%, or 99% thereto.

65. A chimeric or fusion protein according to claim 51, wherein the protein comprises additional amino acid residues to facilitate expression in a recombinant expression system and/or to facilitate purification of the protein.

66. A nucleic acid or vector comprising a nucleic acid encoding a chimeric or fusion protein according to claim 51.

67. A vaccine or immune stimulating composition comprising a chimeric or fusion protein according to claim 51, and optionally, an adjuvant for potentiating an immune response to the chimeric or fusion protein.

68. A method for immunizing a subject against P. gingivalis infection, for treating a P. gingivalis infection in a subject, or for inducing an immune response in a subject to P. gingivalis, the method comprising administering to a subject in need thereof, a chimeric or fusion protein according to claim 51, or a composition or a vaccine or immune stimulating composition comprising the chimeric or fusion protein.

69. The method of claim 68, wherein the immune response is a humoral immune response, or wherein the immune response that is induced comprises a switch from a Th1 to a Th2 immune response.

70. A method according to claim 68, wherein the method further comprises administration of one or more of an antimicrobial compound and an anti-inflammatory agent.

71. A method for obtaining an antibody directed to P. gingivalis, the method comprising administering a chimeric or fusion protein according to claim 51 to a non-human animal, thereby generating antibodies directed to P. gingivalis in the animal.

72. An antibody preparation comprising an antibody directed to P. gingivalis, wherein the antibody preparation is obtained by administering a chimeric or fusion protein, according to claim 51 to a non-human animal, thereby generating antibodies directed to P. gingivalis in the animal, and isolating the antibodies from the animal or an egg thereof.
